(19) United States
(12) Patent Application Publication
(10) Pub. No.: US 2015/0073551 A1
(43) Pub. Date: Mar. 12, 2015

(54) BIOMIMETIC TISSUE GRAFT FOR LIGAMENT REPLACEMENT

(71) Applicant: The UAB Research Foundation, Birmingham, AL (US)

(72) Inventor: Andrew Uehlin, Birmingham, AL (US)

(21) Appl. No.: 14/022,531

(22) Filed: Sep. 10, 2013

(51) Int. Cl. A61L 27/18; A61L 27/38; A61L 27/34; A61L 27/58; A61F 2/08; A61L 27/32

(52) U.S. Cl. CPC A61L 27/18; A61F 2/08; A61L 27/32; A61L 27/34; A61L 27/58; A61L 27/3834; A61L 2430/10; A61L 2420/02; A61L 2420/08; A61L 2400/12
USPC 623/13.12; 427/2.27; 427/2.24

(57) ABSTRACT

Implantable biomimetic ligaments suitable for use in ligament replacement, including, but not limited to, that of the anterior cruciate ligament (ACL) are provided. The replacement implants consist of a biocompatible degradable polymeric scaffold seeded with mesenchymal stem cells (or a combination of different phenotypes). The use of materials such as polylactic acid, fibrin, and nanohydroxyapatite particles, area-dependent compositional modifications, surface topography, biochemical manipulations, and selective growth environments in vitro, allows the scaffold to be populated with the cells mimetic of the native tissue. The mechanical properties of the scaffold support the development of subchondral bone, mineralized fibrocartilage, non-mineralized fibrocartilage, and the ligament proper. The scaffold can be rolled up, transitioning the two-dimensional planar scaffold to a three-dimensional graft for implantation.

### TECHNICAL FIELD

The present disclosure is generally related to biomimetic ligaments suitable for grafting, and to apparatus and methods for the manufacture thereof.

### BACKGROUND

The anterior cruciate ligament (ACL) is the most commonly injured ligament of the knee with over 200,000 patients in the United States diagnosed with ACL injuries annually (Pennisi E. (2002) Science 295: 1011). The ACL is critical to normal kinematics and stability in the knee. It stabilizes the knee joint and controls motion by connecting the femur to the tibia, effectively preventing abnormal types of motion. It prevents dislocation and fracture of bones in the joint by preventing excessive anterior translation of the femur (Beynnon & Fleming (1998) J. Biomech. 31: 519-525).

The ACL is a dense connective tissue comprised of four anatomically distinct areas: the ligament proper, non-mineralized fibrocartilage, mineralized fibrocartilage, and subchondral bone. The ligament proper is an avascular and aneurotic tissue containing an extracellular matrix (ECM) composed mainly of collagen type I and type III, with a low concentration fibroblast cells. As the ligament transcends to its bone insertions, the ligament proper gives way to a region of non-mineralized fibrocartilage. The ECM becomes less dense and regular, with a composition mainly of collagen type I and II. Chondrocytes exist in this matrix and at a much higher concentration than the concentration of fibroblasts in the ligament proper. As the fibrocartilage tissue becomes mineralized, the ECM contains hydroxyapatite (Ca₁₀(PO₄)₆(OH)₂) in addition to collagen. The collagen fibers become more regular and organized parallel to the ligament. Hypertrophic chondrocytes exist in the mineralized fibrocartilage tissue in similar concentrations to the non-mineralized fibrocartilage. The mineralized fibrocartilage increases in hydroxyapatite concentration until it transforms into subchondral bone. This linear variation in cellularity, mineral content and extracellular matrix composition results in a graduated change in stiffness and allows for effective load transfer from ligament to bone, minimizing stress concentrations and preventing failure (Petrigliano et al., (2006) Arthroscopy 22: 441-451). In adults, the ACL has an approximate length of 38 mm and a diameter of 10 mm.

Biomechanical tests performed on the human knees have shown the femur-ACL-tibia complex to tolerate an ultimate load of 2160 (±157) N and have a stiffness of 242 (±28) N/mm. These values correspond with reported ultimate load and a stiffness values for the ACL ligament proper from other sources. Tensile testing on bundles of the ACL ligament have shown it to have a Young's modulus of 516 (±64) MPa (Zantop et al., (2006) Knee Surg. Sports Traumatol. Arthrosc. 14: 982-992; Guilack et al. (2004) Functional Tissue Engineering. New York, N.Y. Springer). Tensile tests on samples of bovine subchondral bone have revealed an ultimate tensile strength of 3.5 (±1.2) GPa. The same tests revealed a Young's modulus of 30 (±7.5) MPa (Braidotti et al., (2000) J. Biomech. 33: 1153-1157). Compression tests on the ACL ligament-to-bone interface revealed compressive moduli of 0.32 (±0.14) MPa and 0.68 (±0.39) MPa for non-mineralized and mineralized fibrocartilage, respectively (Moffat et al., (2009) Clin. Sports Med. 28: 157-176).

Because it is avascular and has a low cell concentration, the natural healing capabilities of the ACL proper are very limited. ACL injury can be treated with physical therapy by strengthening the muscles involved in knee motion, such as the hamstring, quadriceps, calf, hip, and ankle. While physical therapy can return full range of motion, there is an increased risk of further injury. Additionally, an ACL with reduced function will result in increased pressure on the meniscus, resulting in meniscus erosion and an increased risk of osteoarthritis. To avoid these risks and in the case of a severe ACL injury, the torn ligament can (and often must) be permanently replaced (Frank & Jackson (1997) J. Bone Joint Surg. Am. 79: 1556-1576).

Following injury, orthopedic reconstruction of the ACL is often performed to regain normal stability and kinematics. A major challenge in orthopedic reconstruction surgery, however, is the functional integration of soft tissue grafts with subchondral bone. Because integration between graft and bone is essential for musculoskeletal motion, the fixation of these grafts is crucial in the effective repair of injuries to ligaments and tendons (Moffat et al., (2009) Clin. Sports Med. 28: 157-176).

Currently, the implantation of autografts is the most common treatment, with the patellar tendon autograft being the most widely used. The process of harvesting this graft involves the surgical removal of the central one-third of the patellar tendon along with a section of bone from the patella as well as from the insertion point at the tibia. This type of graft is commonly called the “bone-patellar-bone” (BPB) graft. The BPB graft is then fed through a tunnel created by drilling though the tibia, drawn across the knee, and into a tunnel drilled though the femur. The bone section of the graft effectively provides integration into the tibial and femoral tunnels when set with interference screws (Laurencin & Freeman (2005) Biomaterials. 26: 7530-7536). This method is considered as the current “gold standard” despite several disadvantages, the most important being donor site morbidity and danger of recurring instability (Fu et al., (1999) Am. J. Sports Med. 27: 821-830; Fu et al., (2000) Am. J. Sports Med. 28: 124-130).

Another approach of ACL replacement is the use of allografts obtained from cadavers, such as patellar tendon, hamstring tendon, and Achilles tendon (Jackson et al., (1993) The Anterior Cruciate Ligament: Current and Future Concepts. Raven Press, New York, N.Y.). The benefits of using allografts include the lack of a second surgery to harvest the graft, and the elimination of association of donor site morbidity. There are, however, significant disadvantages associated with the use of allografts, such as the potential to transmit disease, cause bacterial infection, elicit an unfavorable immunogenic response from the host, and the inability of the graft to be sterilized without altering the mechanical properties of the tissue (Snook et al., (1983) Clin. Orthop. Relat. Res. 172: 11-13; Miller et al., (1996) Review of Orthopaedics. W.B. Saunders Company, Philadelphia, Pa.).

As a result, an alternative ACL replacement graft is needed and has been heavily investigated. The early use of synthetic prostheses for ACL replacement made of non-resorbable materials such as polyethylene terephtalate (Leeds-Keio Ligament), polypropylene (Kennedy Ligament Augmentation Device), poly(tetrafluoroethylene) (GORE-TEX®), or carbon fibers, was abandoned due to severe inflammatory and foreign body reactions, poor mechanical properties, and very poor durability and abrasion resistance (Laurencin & Freeman (2005) Biomaterials. 26: 7530-7536; Duerselen et al., (1996) Biomaterials 17: 977-982). To overcome these drawbacks, research has shifted towards a biocompatible model and is now focused on tissue-engineered solutions for ACL reconstruction (Laurencin & Freeman (2005) Biomaterials 26: 7530-7536).

### SUMMARY

The present disclosure encompasses embodiments of an artificial prosthetic ligament, and methods of making thereof, advantageous for the replacement of a torn or injured ligament, and in particular of the anterior cruciate ligament (ACL). The compositions of the disclosure provide synthetic scaffolds that replicate to a significant degree the various regions of a native ACL and allow for the colonization of the scaffold by cells that can contribute to the matrix composition and to the mechanical strength of the manufactured ligament.

One aspect of the present disclosure, therefore, encompasses embodiments of a biomimetic composition comprising: a biocompatible scaffold structure comprising a sheet of substantially parallel polymeric microfibers and a population of hydroxyapatite nanoparticles deposited on the sheet, wherein the population of hydroxyapatite nanoparticles can be distributed on the sheet in a pattern mimicking the mineralization of a native ligament to bone enthesis.

In embodiments of this aspect of the disclosure, the biocompatible scaffold structure can be biodegradable.

In embodiments of this aspect of the disclosure, the polymeric microfibers can comprise poly(lactic acid).

In embodiments of this aspect of the disclosure, the biocompatible scaffold structure can further comprise at least one polypeptide deposited thereon.

In embodiments of this aspect of the disclosure, the at least one polypeptide can be selected from the group consisting of: an extracellular matrix polypeptide, fibrin, fibrinogen, a cell growth factor, and a cell differentiation inducer.

In embodiments of this aspect of the disclosure, the at least one polypeptide deposited thereon can be fibrin.

In embodiments of this aspect of the disclosure, the biocompatible scaffold structure can comprise at least two polypeptides deposited thereon, and wherein one polypeptide can be fibrin deposited on the sheet of substantially parallel polymeric microfibers and at least one other polypeptide can be deposited on the fibrin.

In embodiments of this aspect of the disclosure, the biocompatible scaffold structure can further comprise a population of mesenchymal stem cells, or the progeny thereof.

In embodiments of this aspect of the disclosure, the biomimetic composition can comprise: a biocompatible scaffold structure comprising a sheet of substantially parallel polymeric microfibers, a population of hydroxyapatite nanoparticles distributed on the sheet in a pattern mimicking the mineralization of a native ligament to bone enthesis, fibrin deposited on said sheet of polymeric microfibers, at least one polypeptide deposited on the fibrin, wherein the at least one other polypeptide can be selected to promote the growth and/or differentiation of a population of mesenchymal stem cells or progeny thereof colonizing the scaffold structure, and a population of mesenchymal stem cells or progeny thereof, wherein the biocompatible scaffold structure is configured for replacing a native ligament of a subject animal or human.

In embodiments of this aspect of the disclosure, the biocompatible scaffold structure can be configured for replacing a native anterior cruciate ligament.

Another aspect of the disclosure encompasses embodiments of a method of forming a biomimetic scaffold structure, the method comprising the steps of: generating a sheet of substantially parallel polymeric microfibers having polymeric nanofibers deposited on the surface thereof; distributing hydroxyapatite nanoparticles on the sheet in a pattern mimicking the mineralization of a native ligament to bone enthesis; and configuring said sheet for replacing a native ligament of a subject animal or human.

In embodiments of this aspect of the disclosure, the method can further comprise contacting the sheet of substantially parallel polymeric microfibers with an alkali; providing exposed carboxyl groups; decreasing fiber diameter; and increasing surface roughness.

In embodiments of this aspect of the disclosure, the method can further comprise providing fibrin on the surface of the sheet of substantially parallel polymeric microfibers.

In embodiments of this aspect of the disclosure, the method can further comprise the step of colonizing the biomimetic scaffold with a population of mesenchymal stem cells or progeny.

In embodiments of this aspect of the disclosure, the method can further comprise the step of depositing a polypeptide on the fibrin on the surface of the sheet of substantially parallel polymeric microfibers, wherein the polypeptide is selected to promote the growth and/or differentiation of a population of mesenchymal stem cells or progeny thereof colonizing the biomimetic scaffold.

In embodiments of this aspect of the disclosure, the method of distributing hydroxyapatite nanoparticles on the sheet in a pattern mimicking the mineralization of a native ligament can comprise microprinting the hydroxyapatite nanoparticles onto the sheet of substantially parallel polymeric microfibers or electrophoretically depositing the hydroxyapatite nanoparticles, thereby forming a density gradient of the hydroxyapatite nanoparticles mimicking the mineralization of a native ligament to bone enthesis.

### BRIEF DESCRIPTION OF THE DRAWINGS

Further aspects of the present disclosure will be more readily appreciated upon review of the detailed description of its various embodiments, described below, when taken in conjunction with the accompanying drawings.

FIG. 1 schematically shows the variation of four tissue areas on the proposed prosthesis scaffold of the disclosure. The lower graph displays spatially corresponding mineral content to tissue areas.

FIG. 2 schematically illustrates an embodiment of an electrospinning apparatus modified to deposit PLA nanofibers on a rotating drum: 1, Syringe fed by a pump with a charged lead from the voltage source to the needle; 2, polymer solution within the syringe; 3, ejected polymer forming micro- or nanofibers; 4, rotating mandrel; 5, grounding point behind the rotating mandrel; 6, a variable DC voltage supply.

FIG. 3 illustrates an embodiment of an electrospinner for electrospinning a PLA nanofiber mesh on a rotating collector drum.

FIG. 4 illustrates an embodiment of a method of electrospinning PLA nanofibers to form a 100-150 μm mat. A strip over the aligned microfibers prevents electrospun nanofibers from binding to that area.

FIG. 5 is a digital SEM image after EPD of nanoHA after 4 h on an aligned electrospun PCL scaffold, with nanoHA displayed as texture on the fibers.

FIG. 6 schematically illustrates the EPD of nanoHA on the PLA nanofiber mat. A nanoHA gradient is achieved by progressively raising the scaffold out of the solution, as denoted by the arrow.

FIG. 7 schematically illustrates the solution casting of PLA with nanoHA dispersion into a mold, then achieving nanoHA gradient via electrophoresis. Salt will be dispersed after completion of electrophoresis.

FIG. 8 schematically illustrates the solution casting of PLA with dispersed nanoHA and salt into a mold. Arrows indicate the removal of the partition after a partial evaporation of the solution, for bonding of the two casts.

FIG. 9 schematically illustrates a two-dimensional tri-culture model separating three regions of the scaffold with hydrogel dividers, 7.

FIG. 10 schematically illustrates a three-dimensional tissue engineered ACL graft after rolling up a 2-D scaffold.

FIG. 11 illustrates a cross-sectional representation of an electrospun PLA mat indicating 900-1100 μm areas selected for use.

FIGS. 12A-121 illustrate the modification of 24-well tissue culture plates for sample fixation. FIG. 12A: sections of PE tubing for sample fixation. FIG. 12B: Placement of 10 mm risers at the bottom of the well plate used as a frame. FIG. 12C: Placement of the PE tubes from “A” into the frame. FIG. 12D: Application of a biocompatible adhesive to the flat, polished end of the PE tubes. FIG. 12E: Placement of a glass slide with an electrospun PLA sample on four pre-glued PE tubes. FIG. 12F: Transfer of electrospun PLA sample to the glued ends of the four PE tubes. FIG. 12G: Separation of the PLA-capped tubes. FIG. 12H: PLA-capped tube before (left) and after (right) trimming of excess PLA. FIG. 12I: Placement of the PLA-capped tube face-down into a new, sterile 24-well culture plate.

FIGS. 13A-13C are a series of digital SEM images of electrospun PLA fibers using the following uptake rates: FIG. 13A: Stationary; FIG. 13B: 3500 rpm; FIG. 13C: 7000 rpm. Magnification=1000×. Scale bars=25 μm.

FIG. 14 is a bar graph illustrating the frequency distribution of electrospun PLA fiber diameter at 0 rpm (stationary), 3500 rpm, and 7000 rpm uptake rates.

FIG. 15 is a graph illustrating the percent reduction in area of 7000 rpm PLA nanofibers from various NaOH treatment times. Values were expressed as mean±S.E.M. Percent reduction in area is dependent on NaOH treatment time and displays a logarithmic trend, r²=0.9816.

FIG. 16 is a graph illustrating fiber diameter of various uptake rates and NaOH treatment times on electrospun PLA. Values were expressed as mean±S.E.M. NaOH treatments performed on 7000 rpm fibers.

FIGS. 17A-17F are a series of digital SEM images of electrospun PLA fibers subjected to the following NaOH treatment times: FIG. 17A: 0 min (control); FIG. 17B: 1 min, arrow indicates minor surface roughness; FIG. 17C: 5 min, arrows indicate visible surface roughness and pitting at the fiber interface; FIG. 17D: 10 min, arrow indicates interface pitting; FIG. 17E: 20 min, arrows indicate significant surface roughness and pitting at the fiber interface; FIG. 17F: 40 min, arrow indicates significant pitting and visibly decreased fiber diameter. Magnification=50,000×. Scale bars=1 μm.

FIG. 18 is a graph illustrating the average percent swelling of the PLA fibers in the untreated and NaOH-treated conditions over 60 d. Values were expressed as mean±S.E.M.

FIGS. 19A and 19B are a pair of digital SEM images of electrospun PLA fibers subjected to fibrin immobilization in: FIG. 19A: untreated condition, arrow indicates a particle of (presumably) fibrin found randomly throughout the sample; FIG. 19B: 20 min NaOH treatment, arrows indicate connecting membranes of fibrin. Magnification=50,000×. Scale bars=1 μm.

FIG. 20 is a bar graph illustrating cellular proliferation over 14 d. There was an overall increase in proliferation at day 7, followed by a step-wise increase manifesting at day 14. Values expressed as mean±S.E.M. (**p<0.01).

FIG. 21 is a graph illustrating gene expression profile for ALP over 14 d. At day 14, ALP expression was greatest in the NaOH-treated PLA+fibrin samples. Values were expressed as mean±S.E.M. (**p<0.01).

FIGS. 22A-22F are a series of digital SEM images of the hMSCs on the PLA samples at day 14 at 250× in BSE mode (left images) and 10,000× in SE mode (right images). FIGS. 22A and 22B: Untreated PLA; FIGS. 22C and 22D: 20 min NaOH-treated PLA; and FIGS. 22E and 22F: 20 min NaOH-treated PLA+fibrin. Scale bar (left images)=200 μm, (right images)=5 μm.

FIG. 23 is a graph illustrating treatment-dependent variations of the stress-strain curves observed during mechanical testing of the PLA samples. (A) yield stress, (B) maximum stress, (C) strain at yield stress, (D) strain at failure, calculated as the intersection of a 45° line tangent to the stress-strain curve.

FIG. 24 is a graph illustrating yield stress of the aligned PLA fibers with various surface treatments, compared to natural ligament/graft tissue. Values were expressed as mean±S.E.M. (PT=patellar tendon, “Ligament” includes ACL, PCL, and LCL).

FIG. 25 is a graph illustrating maximum stress of the aligned PLA fibers with various surface treatments, compared to natural ligament/graft tissue. Values were expressed as mean±S.E.M. (PT=patellar tendon, “Ligament” includes ACL, PCL, and LCL).

FIG. 26 is a graph illustrating the percent strain at failure of the aligned PLA fibers with various surface treatments, compared to natural ligament/graft tissue. Values were expressed as mean±S.E.M.

FIG. 27 is a graph illustrating the elastic modulus of the aligned PLA fibers at various temperatures, compared to natural ligament/graft tissue. Values were expressed as mean±S.E.M. (PT=patellar tendon, “Ligament” includes ACL, PCL, and LCL).

FIG. 28 is a graph illustrating an overlay of the stress-strain profiles from a control, 20 min NaOH-treated sample, and 20 min NaOH-treated sample with fibrin at body temperature. Dotted lines indicate the stress-strain profiles of the ACL for comparison.

The drawings are described in greater detail in the description and examples below.

The details of some exemplary embodiments of the methods and systems of the present disclosure are set forth in the description below. Other features, objects, and advantages of the disclosure will be apparent to one of skill in the art upon examination of the following description, drawings, examples and claims. It is intended that all such additional systems, methods, features, and advantages be included within this description, be within the scope of the present disclosure, and be protected by the accompanying claims.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

As used herein, the following terms have the meanings ascribed to them unless specified otherwise. In this disclosure, “comprises,” “comprising,” “containing” and “having” and the like can have the meaning ascribed to them in U.S. patent law and can mean “includes,” “including,” and the like; “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially” of or “consists essentially” or the like, when applied to methods and compositions encompassed by the present disclosure have the meaning ascribed in U.S. patent law and the term is open-ended, allowing for the presence of more than that which is recited so long as basic or novel characteristics of that which is recited is not changed by the presence of more than that which is recited, but excludes prior art embodiments.

Prior to describing the various embodiments, the following definitions are provided and should be used unless otherwise indicated.

### ABBREVIATIONS

ACL, anterior cruciate ligament; ECM, extra cellular matrix; BPB, bone-patellar-bone; PLA, poly(lactic acid); PCL, poly(caprolactone); HA, hydroxyapatite; nanoHA, hydroxyapatite nanoparticles; PTFE, poly(tetrafluoroethylene); EPD, electrophoretic deposition; mmP13, matrix metalloprotease-13; IGF-1, insulin growth factor-1; CTGF, connective tissue growth factor; FGF, fibroblast growth factor; HDACs, histone deacetylases; TGF, transforming growth factor; BMP, bone morphogenic protein; H & E, hematoxylin and eosin; BMPR, bone morphogenetic protein receptor; BAP, bone-specific alkaline phosphatase; RT-PCR, real time polymerase chain reaction; UUMC, uniaxial unconfined micro-compression; DIC, digital image correlation; FE, finite element; hMSCs, human mesenchymal stem cells.

### DEFINITIONS

In describing and claiming the disclosed subject matter, the following terminology will be used in accordance with the definitions set forth below.

The term “biomimetic” as used herein refers to a material or structure designed to resemble and/or function in a manner similar to a structure, organ, or tissue found in a native state in an animal or human. In the embodiments of the disclosure, the biomimetic compositions herein disclosed are suitable for the replacement structurally and functionally of a ligament such as, but not limited to, an ACL.

The term “biocompatible” as used herein refers to a material that does not elicit any undesirable local or systemic effects in vivo.

The term “scaffold” as used herein refers to a material that can provide a supporting structure on which animal cells may attach and proliferate. The scaffold may be configured to resemble the shape and size of a native animal or human structure or physical feature that it is desired to replace.

The term “substantially parallel” as use herein refers to the arrangement of microfibers wherein at least 80%, preferably at least 85%, more preferably at least 90%, more preferably at least 95%, and most preferably at least 99% of the microfibers are parallel to one another.

The term “extracellular matrix polypeptide” as used herein refers to a polypeptide found in the extracellular matrix of an animal or human tissue including, but not limited to, a fibrous protein, a glycosaminoglycan, heparan sulfate, chondroitin sulfate, keratan sulfate, collagen, elastin, fibronectin, laminin, and the like.

The term cell growth factor as used herein refers to a naturally occurring substance capable of stimulating cellular growth, proliferation and cellular differentiation. Usually it is a protein or a steroid hormone. Growth factors are important for regulating a variety of cellular processes. Growth factors typically act as signaling molecules between cells. Examples are cytokines and hormones that bind to specific receptors on the surface of their target cells. They often promote cell differentiation and maturation, which varies between growth factors. For example, bone morphogenic proteins stimulate bone cell differentiation, while fibroblast growth factors and vascular endothelial growth factors stimulate blood vessel differentiation (angiogenesis). While growth factor implies a positive effect on cell division, cytokine is a neutral term with respect to whether a molecule affects proliferation. Some cytokines can be growth factors, such as G-CSF and GM-CSF. Individual growth factor proteins tend to occur as members of larger families of structurally and evolutionarily related proteins such as, but not limited to, Adrenomedullin (AM), Angiopoietin (Ang-2), Autocrine motility factor, Bone morphogenetic proteins (BMP-2, BMP-4, BMP-6), Brain-derived neurotrophic factor (BDNF), Epidermal growth factor (EGF), Erythropoietin (EPO), Fibroblast growth factor (FGF-2 (FGF-β), FGF-4), Glial cell line-derived neurotrophic factor (GDNF), Granulocyte colony-stimulating factor (G-CSF), Granulocyte macrophage colony-stimulating factor (GM-CSF), Growth differentiation factor-9 (GDF-5, GDF-7, GDF-8, GDF9, GDF-11), Hepatocyte growth factor (HGF), Hepatoma-derived growth factor (HDGF), Insulin-like growth factor (IGF-1, IGF-2), Migration-stimulating factor, Myostatin (GDF-8), Platelet-derived growth factor (PDGF), Thrombopoietin (TPO), Transforming growth factor-alpha (TGF-α), Transforming growth factor-beta (TGF-β), Tumor necrosis factor-alpha (TNF-α), and Vascular endothelial growth factor (VEGF).

### DESCRIPTION

The human anterior cruciate ligament (ACL) is ruptured over 200,000 times per year (or an incidence of 1 in 3000) in the United States, resulting in over $1 billion of medical costs annually. The current gold standard for surgical repair is the patellar tendon autograft, but this treatment is far from optimal due to lengthy recovery time, the potential for developing arthritis, associated donor site morbidity, and degenerative joint disease. These limitations have prompted the development of a tissue-engineered solution. However, many attempts at creating an ACL replacement graft have failed due to issues with inhomogeneous cellular infiltration and lack of surface penetration though the scaffold, the lack of multiple cellular phenotypes mimetic of native ACL tissue, poor mechanical properties, and poor biocompatibility.

The implantable devices of the disclosure are engineered biomimetic ligament grafts suitable for use in ligament replacement, including, but not limited to, that of the anterior cruciate ligament (ACL). The replacement implants can consist of a biocompatible degradable polymeric scaffold seeded with mesenchymal stem cells (or a combination of different phenotypes). The scaffold can be effectively populated with the appropriate cells mimetic of the native tissue by the use of selected materials such as, but not limited to, poly(lactic acid), fibrin, and nanohydroxyapatite (nanoHA) particles, area-dependent compositional modifications, surface topography, biochemical manipulations, and selective growth environments in vitro. The mechanical properties of the scaffold support the development and linear progression of four anatomically distinct tissue areas: subchondral bone, mineralized fibrocartilage, non-mineralized fibrocartilage, and the ligament proper. Following cellular confluence on the surface of the scaffold, it can be rolled up, transitioning the two-dimensional scaffold to a three-dimensional graft, prior to implantation. Optionally, the scaffold can be placed in a bioreactor to allow the cells within the scaffold to further bind to each other and the scaffold material. The finished constructs resemble anatomical and mechanical properties of such as the native human ACL. The device can then be surgically implanted in a similar manner to, for example, a patellar tendon graft during ACL reconstruction surgery, eliminating the need for secondary surgery to obtain an autograft, or the use of an allograft.

The disclosure provides embodiments of a two-dimensional scaffold biomimetic for replacement of a ligament such as, but not limited to, the ACL. In the mimetic ligaments of the disclosure, cellular confluence on the scaffold can be obtained before rolling the scaffold up into a three-dimensional structure, and thereby ensuring true cellular penetration throughout the entirety of the graft. The materials chosen are fully biocompatible and degradable; as the cells spread out and multiply within the scaffold, the synthetic scaffold material is broken down and replaced by cells and cell-derived material. Similarly, the surface topography of the scaffold encourages direction cell growth in specific regions, providing mechanical properties similar to the native tissue.

Tissue engineering of a ligament such as an ACL requires cells that are capable of producing a ligament-like extracellular matrix and a degradable, thee-dimensional scaffold that supports tissue development with close mechanical properties to the native structure. Furthermore, the degradable scaffold must exhibit biocompatibility and high interconnecting porosity to promote cell attachment, ingrowth, and differentiation. Accordingly, the biomimetic structures of the disclosure comprise a biocompatible and degradable polymeric scaffold seeded with mesenchymal stem cells (or a combination of different cell phenotypes) that can effectively populate the scaffold though the use of ligament-specific materials, area-dependent compositional modifications, surface topography, biochemical manipulations, and selective growth environments in vitro. Embodiments of the scaffold can be engineered to provide four anatomically distinct tissue areas: subchondral bone, mineralized fibrocartilage, non-mineralized fibrocartilage, and the ligament proper, to create a cell-populated, biomimetic composition of appropriate size, shape, and mechanical function for the ligament to be replaced.

A desirable property of any device or prosthetic implanted in the body is the selection of materials that are biocompatible, i.e. biologically inert materials that can be integrated into the body and do not elicit an adverse immunogenic response. For an engineered ligament graft such as an ACL graft, the mechanical properties of the materials desirably should resemble those of the native ligament to effectively provide similar biomechanical function. Furthermore, it is most advantageous that post-surgical graft-to-bone enthesis be established, so that the graft material is able to integrate into bone to facilitate adequate anatomical function.

A variety of biocompatible and degradable materials of natural and synthetic origin have been studied, including collagen type I (Dunn et al., (1995) J. Biomed. Mater. Res. 29: 1363-1371; Caruso & Dunn (2005) J. Biomed. Mater. Res. A. 73: 388-397), hyaluronic acid (Cristino et al., (2005) J. Biomed. Mater. Res. A. 73: 275-283), silk (Altman et al., (2002) Biomaterials 23: 4131-4141; Chen et al., (2003) J. Biomed. Mater. Res. A. 67: 559-570), and resorbable polymers such as poly(lactic acid) (PLA) and poly(caprolactone)(PCL) (Hasegawa et al., (1999) Clin. Orthop. Relat. Res. 358: 235-243; Lu et al., (2005) Biomaterials 26: 4805-4816; Thomas et al., (2006) J. Nanosci. Nanotechnol. 6: 487-493). Collagen and hyaluronic acid degrade too rapidly to establish adequate cell ingrowth and lack sufficient desirable mechanical properties to be used as a scaffold for such as an ACL graft. Silk, PLA, and PCL, however, have more suitable characteristics as a scaffold material, with slower degradation rates and desired mechanical properties (Laurencin & Freeman (2005) Biomaterials 26: 7530-7536; Altman et al. (2002) Biomaterials, 23; 4131-4141; Duerselen et al. (2001) J. Biomed. Mater. Res. 58: 666-6720).

A variety of polymers can be modified to obtain functional properties and design flexibility desirous in a scaffold. Similarly, biodegradability can be achieved by tailoring some of these polymers (Murugan & Ramakrishna (2007) Tissue Eng. 13: 1845-1866). As such, embodiments of biomimetic ligaments of the disclosure may advantageously comprise such as poly(lactic acid), poly(caprolactone), or a combination thereof. The biomimetic ligaments further comprise dispersed nano-hydroxyapatite particles in a poly(lactic acid) matrix of the disclosure.

Poly(lactic acid) (PLA) as used herein refers to an aliphatic polyester derived from renewable resources, such as corn starch or sugarcane. It is a biodegradable thermoplastic, and the degradation product, lactic acid, is metabolically innocuous, making it an advantageous material for medical applications. As such, it is one of the few biodegradable polymers approved for human clinical use.

The degradation of PLA involves random hydrolysis of its ester bonds to form lactic acid that enters the tricarboxylic acid cycle to be excreted as water and carbon dioxide. The degradation rate can vary by altering factors such as structural configuration, morphology, stresses, crystallinity, molecular weight, copolymer ratio, amount of residual monomer, porosity and site of implantation, and the like, by methods well known to those in the art.

Another suitable polymer for use in the scaffolds of the disclosure is poly(caprolactone) (PCL) derived by chemical synthesis from petroleum. It is a semi-crystalline, resorbable, aliphatic polyester that biodegrades by hydrolysis of ester linkages and eventual intracellular phagocytosis. PCL degrades at a lower rate than PLA and is useful in long term, implantable drug delivery systems.

The biomimetics of the disclosure further incorporate hydroxyapatite (HA), which is the main inorganic mineral present in animal teeth and bones. It is biocompatible, degradation-resistant and highly osteotropic as it is one of the few biomaterials able to establish a substantial continuity between itself and bone. Nanohydroxyapatite (NanoHA) particles have been incorporated into polymer tissue constructs using various tissue fabrication techniques such as electrospinning and solution or melt casting, resulting in a significant increase in mechanical properties and improved cell binding and proliferation, resulting in improved biointegration. In nanoscale synthetic form with a similar chemical composition to that of bone, nanoHA can be bound in vivo to a great variety of molecules such as enzymes and proteins. Accordingly, growth factors can be bound to nanoHA particles incorporated into the biomimetic ligament grafts of the disclosure to tailor cell integration and differentiation.

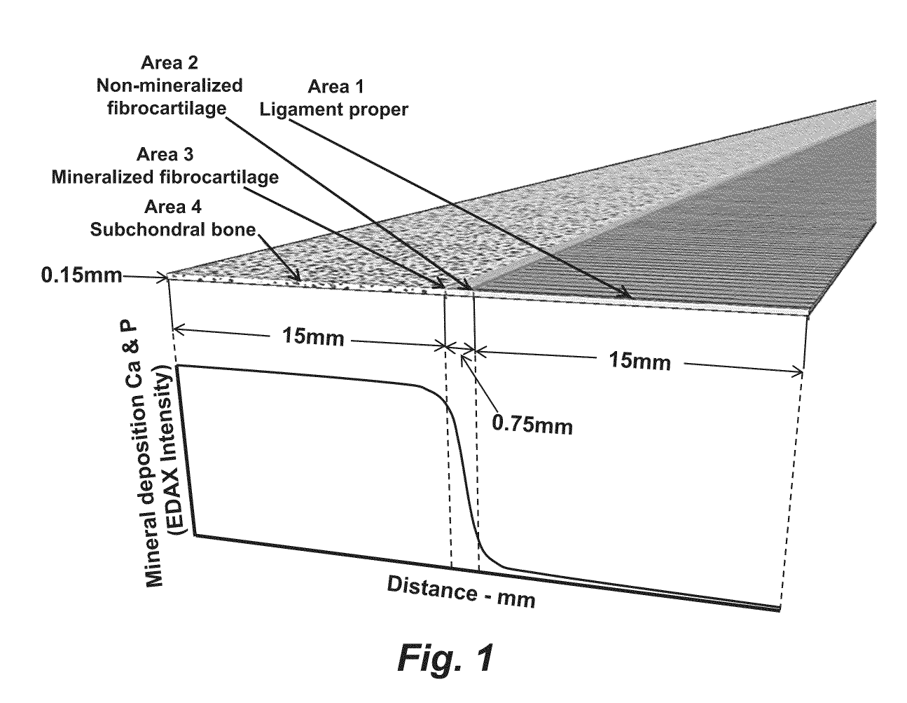

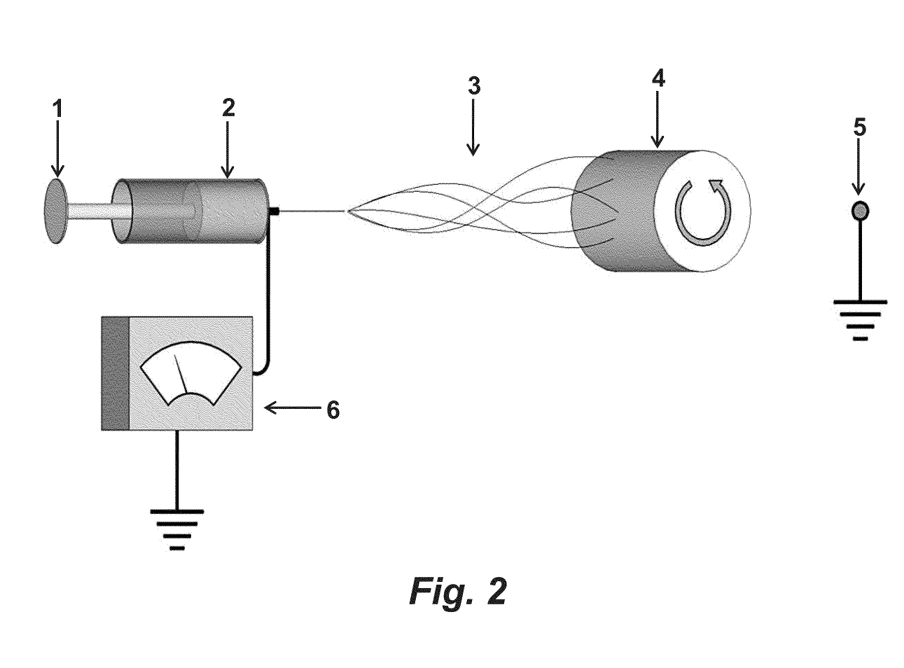

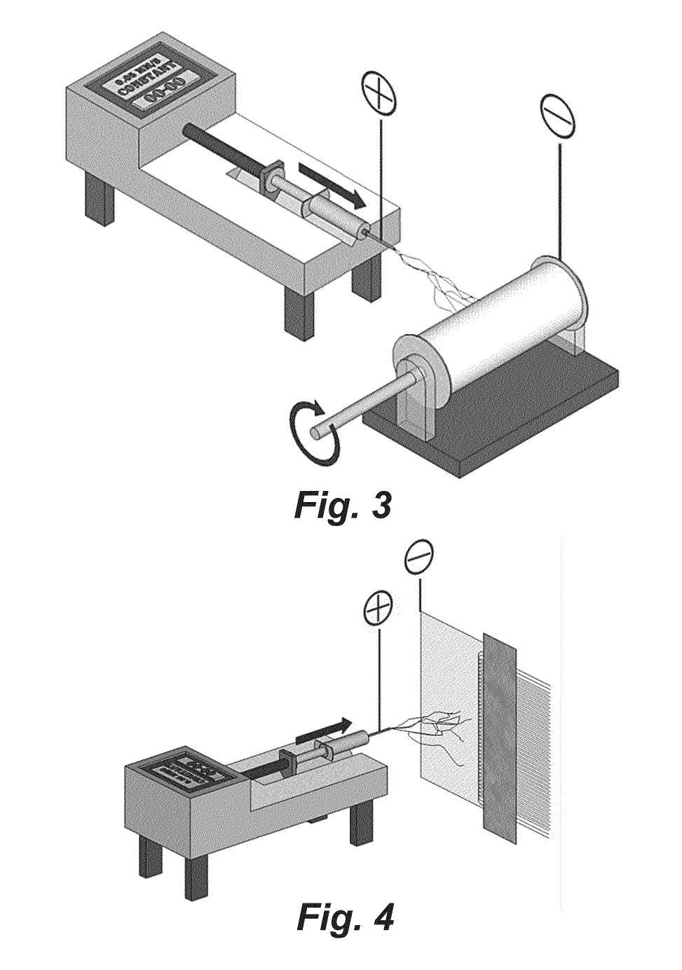

The biomimetic ligaments of the disclosure provide scaffold structures that resemble and facilitate the development of the four tissue types found in ligaments such as the ACL and ACL-to-bone interface, as illustrated in FIG. 1. The morphology of the scaffolds of the disclosure, accordingly, spatially correspond to these tissue types over four distinct areas: the ligament proper (area 1), non-mineralized fibrocartilage (area 2), mineralized fibrocartilage (area 3), and subchondral bone (area 4). This material can be formed by such as a modified electrospinning device as schematically shown in FIG. 2.

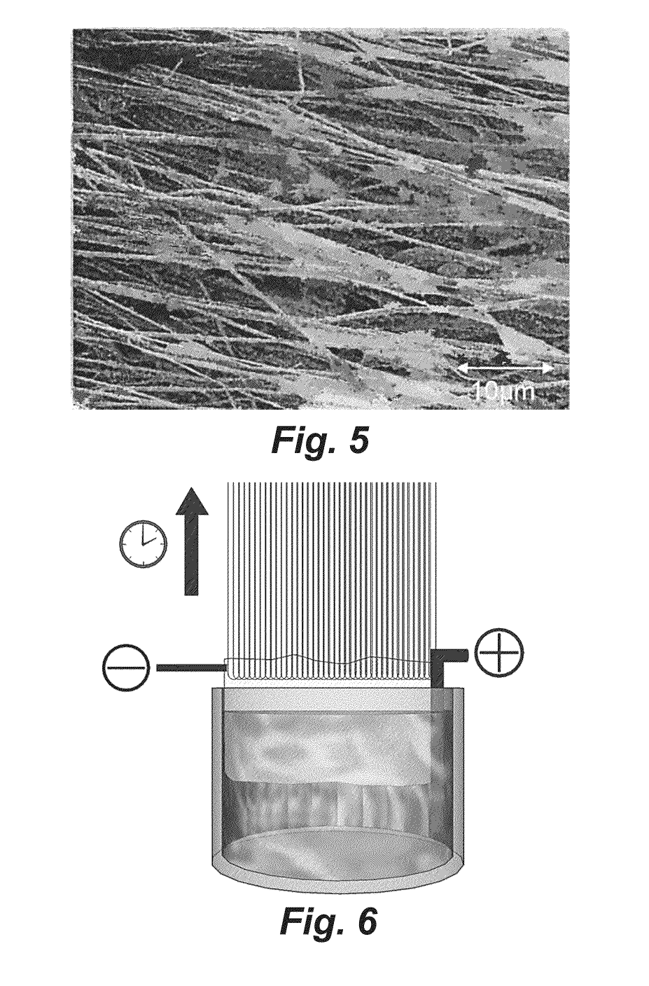

Electrospinning formation of a nanofiber PLA mat can be followed by the incorporation or deposit of nanoHA onto the mat via such as electrophoretic deposition (EPD). EPD is a two-step process that allows rapid deposition rates with a high degree of control over deposition thickness. First, charged colloidal-sized hydroxyapatite nanoparticles (nanoHA) of, but not limited to, between about 0.2 μm to about 40 μm in suspension migrate towards a counter-charged electrode at which deposition occurs. Secondly, these particles are deposited (discharged and flocculated) on to the substrate, as described by Wei et al., (2005) J. Mater. Sci. Mater. Med. 16: 319-324, incorporated herein by reference in its entirety, and as shown in FIG. 5.

A nanoHA gradient may be generated in a scaffold according to the disclosure by progressively raising the nanofiber mat out of the solution during the EPD process. The deposition thickness depends on the time allowed for the EPD process, and the deposition gradient will depend on the rate of raising the nanofiber mat out of the solution, as illustrated in FIG. 6.

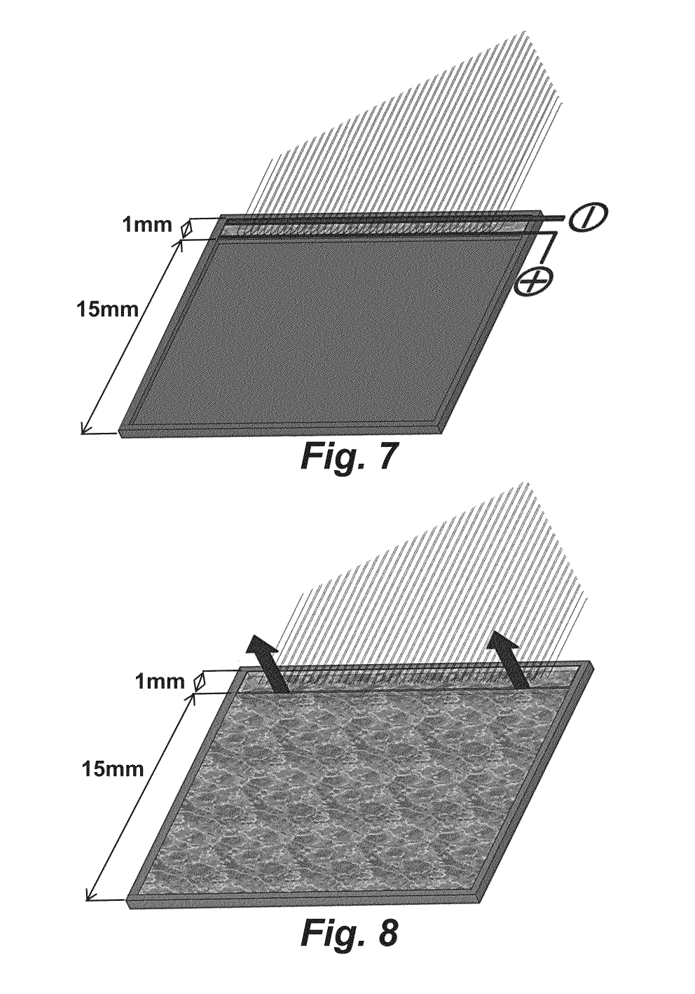

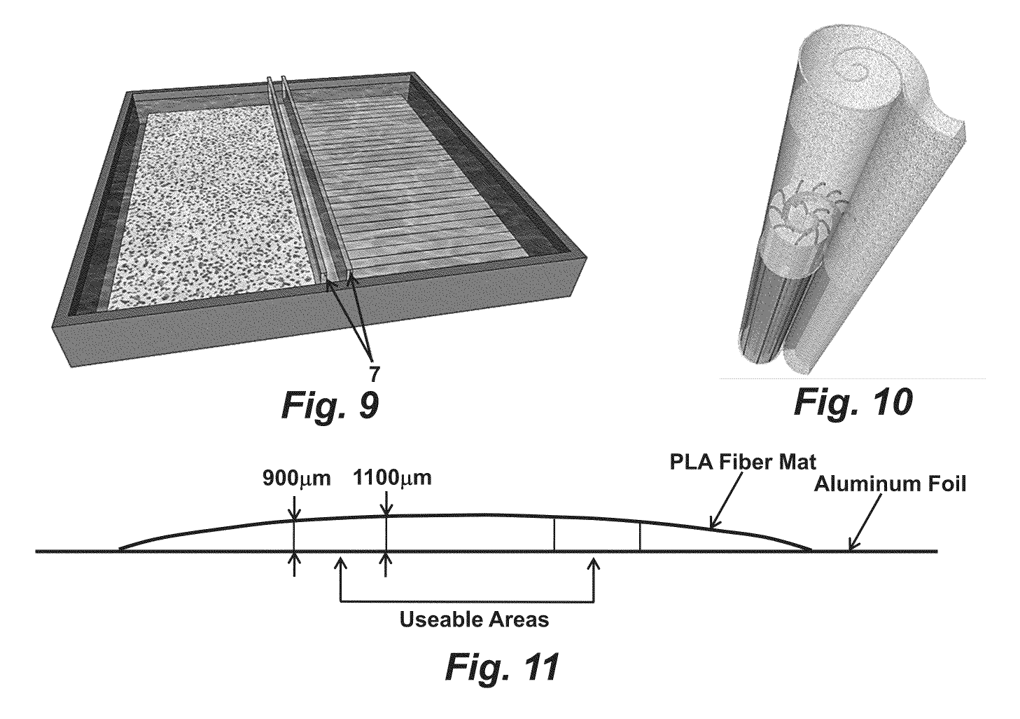

Another aspect of the disclosure provides embodiments of a cast PLA film. A first manufacturing step involves casting to produce a thin porous PLA film with a dispersion of nanoHA particles over a distally increasing gradient relative to the microfibers. This can be accomplished by preparing a solution of PLA and nanoHA and then casting the solution into a section of a mold partitioned by a thin, inert film with desired dimensions such as, but not limited to, about 15 mm height and about 100 to about 150 μm in thickness. While still in the solution state, a nanoHA gradient can be formed via electrophoresis. Then an even dispersion of granular salt (approximately 100 μm) can be incorporated into the solution, followed by overlaying a microfiber mat of the disclosure onto the 1 mm casting, as shown in FIG. 7. A second step then involves casting a solution of PLA, granular salt, and evenly dispersed nanoHA (with a greater concentration than the previous PLA-nanoHA solution) into the remaining mold space, as shown in FIG. 8.

The solvent can be allowed to partially evaporate until a viscous solution remains, and then the partition may be removed, allowing the two separate casts to bond. The remaining solvent can be evaporated, and the resulting film removed from the mold and washed out to remove the salt and residual solvent. By selectively using salt granules with close dimensions to the thickness of the cast film, porosity can be obtained through the film.

Due to its hydrophobic nature and smooth morphology, the surface of PLA alone is not an ideal substrate for cellular integration and the subsequent synthesis of new tissue. The use of NaOH to introduce carboxyl groups and increase surface roughness can assist in the effective binding of cells, but the combination of the surface carboxyl groups with an intermediary protein (fibrin) has a potent effect on cellular attachment and proliferation.

Fibrin is a natural polymer in the human body that is critical for hemostasis and wound healing. Fibrin can be created ex vivo though the rapid enzymatic polymerization of fibrinogen with thrombin from either allogeneic or autologous sources. Due to a natural binding affinity, the immobilization of many growth factors as well as improved cell seeding efficiency and uniformity of cell distribution is possible (Ahmed et al., (2008) Tissue Eng. Part B Rev. 2008; Miller et al., (2009) Comb. Chem. High Throughput Screen 12: 604-618; Peng et al., (2004) Blood 103: 2114-2120; Schense & Hubbell (1999) Bioconjug. Chem. 10: 75-81). Furthermore, the biocompatibility and ease of processing from autologous sources eliminates immunological concerns (Cummings et al., (2004) Biomaterials 25: 3699-3706).

A combination of atomic force microscopy (AFM) and fluorescence microscopy was used to measure the strain at failure (extensibility) of fibrin (Liu et al., (2006) Science 313: 634). The strain at failure was high, with 332% and 226% elongation in the cross-linked and uncross-linked states, respectively, indicating that these properties of fibrin can affect the overall mechanical resilience of PLA when immobilized on the surface. Accordingly, the effects of stepwise surface degradation and morphological modifications on the mechanical properties of highly-aligned electrospun PLA nanofibers were investigated. Surface morphology and fiber diameter were altered though induced hydrolytic degradation via NaOH treatments over various time periods and then mechanical properties were evaluated under a tensile load and compared to natural ligament and ligament-replacement graft materials from literature. Fibrin was immobilized on the surface of a NaOH treated sample, which was then re-evaluated under tensile load at room temperature (21° C.) and body temperature (37° C.) to assess the effects of the fibrin and the combined effects of elevated temperature and fibrin on the mechanical properties.

The mechanical studies of unidirectionally electrospun PLA nanofiber mats of the disclosure following various stepwise surface treatments were investigated. Induced hydrolytic degradation of the fibers in 0.25M NaOH for various time periods followed by immobilization of fibrin on the hydrolyzed fiber surfaces were shown to significantly affect yield stress, maximum stress, and strain at failure. The combination of 20 min of NaOH hydrolysis followed by the surface immobilization of fibrin effectively altered the mechanical properties to values within the range of human ligament and current ligament-replacement graft materials. Furthermore, mechanical tests performed at 37° C. on a fibrin-coated NaOH-treated sample further indicated a stress-strain profile closer to human ACL tissue than the other treated/untreated samples. The elastic modulus and strain at yield stress were not significantly affected by the various surface treatments, and the values reported at 37° C. were considerably higher (approximately 2.5×) than the upper range of human ligament and ligament-replacement graft materials. These properties are inherent of the molecular weight of the polymer used and thus can be altered during synthesis. The most advantageous combination of fiber orientation/alignment, induced hydrolytic degradation, and immobilization of fibrin can result in modification of the mechanical properties of an electrospun PLA tissue scaffold, so as to be mechanically mimetic of human ligament and ligament-replacement graft tissue.

Embodiments of biomimetic ligaments of the disclosure can advantageously promote the differentiation of mesenchymal stem cells (MSCs). Since the ligaments of the disclosure are composed of four differing tissue areas, each area requires a unique biochemical approach that considers three factors: growth factors, retroviral gene infection, and adhesive ligands. These three factors can be considered where appropriate for the four tissue areas that comprise a ligament such as an ACL.

### Area 1: The Ligament Proper

The ligament proper advantageously has a high degree of fibroblast differentiation and deposition of collagen type I and III. These factors may be incorporated though the addition of connective tissue growth factor (CTGF). CTGF has effectively been shown to induce MSC differentiation into fibroblasts. Research indicates that CTGF-stimulated MSCs lose surface mesenchymal epitopes, express broad fibroblastic characteristics, and increase synthesis of collagen type I and III (Lee et al., (2010) J. Clin. Invest. 120: 3340-3349; Wang et al., (2006) Circulation 114: 1200-1205). As an added benefit, the CTGF/MSC derived fibroblasts exhibit diminished abilities to differentiate into non-fibroblastic lineages including osteoblasts, chondrocytes and adipocytes (Lee et al., (2010) J. Clin. Invest. 120: 3340-3349).

To further stimulate the high levels of type I collagen deposition required in the ligament, the use of insulin growth factor-1(IGF-1), a fibroblast-interacting growth factor that selectively induces type I collagen deposition, can be incorporated in addition to CTGF (Miller et al., (2009) Comb. Chem. High Throughput Screen 12: 604-618; Jonsson et al., (1993) Bioscience Reports 13: 297-302).

### Area 2: Non-Mineralized Fibrocartilage

In area 2, the MSCs must differentiate into hypertrophic chondrocytes. Transforming growth factor beta-1(TGF-β1) can be used to achieve this differentiation path. TGF-β1 has been shown in studies to induce chondrogenic differentiation of MSCs, with production of cartilage specific proteoglycans and cartilage type II (Park et al., (September 2010) Biomaterials; Augello & De Bari (2010) Hum. Gene Ther. 21: 1226-1238; Buxton et al., (2010) Tissue Eng. Part A). The TGF-β family of growth factors has successfully been used and is a key requirement in in vitro chondrogenic assays (Augello & De Bari (2010) Hum. Gene Ther. 21: 1226-1238; Danisovic et al., (2009) Gen. Physiol. Biophys. 28: 56-62; Salinas et al., (2007) Tissue Eng. 13: 1025-1034; Lim et al., (2010) J. Materials Sci.: Materials in Medicine 21: 2593-2600). The deposition of type I collagen can be stimulated in the same manner as previously described for area 1 through the use of IGF-1.

### Area 3: Mineralized Fibrocartilage

Mineralized fibrocartilage requires type X collagen deposition and the differentiation of MSCs into hypertrophic chondrocytes. The use of bone morphogenic proteins (BMPs) can be used to facilitate this response. Within the TGF superfamily, BMPs offer potential options to regulate tissue development for area 3, as they have been identified to induce osteogenesis as well as promote both chondrogenesis and chondrocyte hypertrophy (Chen et al., (2004) Growth Factors 22: 233-2341; Miller et al., (2009) Comb. Chem. High Throughput Screen 12: 604-618; Volk et al., (1998) J. Bone Miner. Res. 13: 1521-1529; Shen et al., (2010) J. Cell Biochem. 109: 406-416; Takemoto et al., (2010) J. Orthop. Trauma 24: 564-566; Zhou et al., (August 2010) Int Orthop.). The use of BMP-7 has been shown to induce both chondrogenesis or osteogenesis, express collagen type X, and decrease the expression of collagen type I (Zhou et al., (August 2010) Int Orthop.). To inhibit the osteogenic differentiation potential of BMP-7 and promote chondrogenic differentiation of the MSCs, it can be advantageous to incorporate TGF-β1. Studies show that in the presence of TGF-β1, BMP-7 promotes chondrogenic rather than osteogenic differentiation (Shen et al., (2010) J. Cell Biochem. 109: 406-416; Miyamoto et al., (2007) J. Orthopaedic Sci. 12: 555-561). The presence of hydroxyapatite nanoparticles on the scaffold can provide the mineralization of area 3 mimetic of a native bone-ligament interface.

### Area 4: Subchondral Bone

Area 4 requires the formation of subchondral bone; therefore, cell differentiation into osteoblasts is required. Furthermore, it requires type X collagen deposition and inhibition of type I collagen. However, many growth factors for bone induce collagen deposition of all types. Because it is desired to selectively induce bone only in this area, a specific growth factor or group of growth factors is required. As with area 3, the BMP family of growth factors can be advantageously used. Two BMP growth factors can serve the purposes of area 4 optimally: BMP-2, which selectively induces osteogenesis, and BMP-7, to promote osteogenesis and formation of collagen type X while inhibiting collagen type I (Chen et al., (2004) Growth Factors 22: 233-241; Volk et al., (1998) J Bone Miner. Res. 13: 1521-1529; Takemoto et al., (2010) J. Orthop. Trauma 24: 564-566). Additionally, incorporating hydroxyapatite nanoparticles on the scaffold can further induce accelerated osteogenesis in combination with BMP-7 and BMP-2 (Li et al., (September 2010) J Biomed. Mater. Res. A).

### Additional Modifications

If necessary, vascular endothelial growth factor (VEGF) can be incorporated into the entirety of the scaffold to promote angiogenesis; this can be determined following in vitro studies. Similarly, the biomimetic peptide, RGD can be attached to the scaffold, with the goal of enhancing cell/material associations. These mimetic peptides can facilitate cell adhesion by engaging cell surface integrin receptors (Hennessy et al., (2009) Biomaterials 30: 1898-1909; Anderson et al., (2009) Biomacromolecules 10: 2935-2944).

As previously stated, many growth factors can be immobilized onto fibrin via a natural binding affinity without the need for covalent cross-linking or chemical modification (Miller et al., (2009) Comb. Chem. High Throughput Screen 12: 604-618; Peng et al., (2004) Blood 103: 2114-2120). It has been shown that growth factors containing heparin-binding domains bind to fibrin as well. This is verified by research indicating fibroblast growth factor-2 (FGF-2) having high affinity binding domains for fibrin and heparin (Peng et al., (2004) Blood 103: 2114-2120). Similarly, members of the TGF superfamily such as TGF-β1, BMP-2 also demonstrated adequate binding with fibrin and heparin. IGF-1 can be engineered with a heparin binding domain that has been shown to effectively bind to fibrin (Campbell et al., (1999) J. Biol. Chem. 274: 30215-3021).

CTGF can be bound to fibrin though intermediary binding with fibronectin (Yoshida & Munakata (2007) Biochim. Biophys. Acta 1770: 672-680). It is therefore postulated that multiple combinations of growth factors can be selectively bound to a fibrin-coated scaffold over specific spatial domains.

Transitioning the 2-D Scaffold into a 3-D Graft

Following the appropriate surface modifications, hMSCs can be cultured onto the scaffold and allowed to differentiate and thereby produce a variable ECM spatially corresponding to the aforementioned bioactive areas. The scaffold can then be rolled up to the proper diameter creating a 3-D ligament graft with hierarchical ECM and phenotype variation (including bone fixations at either end), corresponding to natural ligament and a ligament-replacement graft.

One aspect of the present disclosure, therefore, encompasses embodiments of a biomimetic composition comprising: a biocompatible scaffold structure comprising a sheet of substantially parallel polymeric microfibers and a population of hydroxyapatite nanoparticles deposited on the sheet, wherein the population of hydroxyapatite nanoparticles can be distributed on the sheet in a pattern mimicking the mineralization of a native ligament to bone enthesis.

In embodiments of this aspect of the disclosure, the biocompatible scaffold structure can be biodegradable.

In embodiments of this aspect of the disclosure, the polymeric microfibers can comprise poly(lactic acid).

In embodiments of this aspect of the disclosure, the biocompatible scaffold structure can further comprise at least one polypeptide deposited thereon.

In embodiments of this aspect of the disclosure, the at least one polypeptide can be selected from the group consisting of: an extracellular matrix polypeptide, fibrin, fibrinogen, a cell growth factor, and a cell differentiation inducer.

In embodiments of this aspect of the disclosure, the at least one polypeptide deposited thereon can be fibrin.

In embodiments of this aspect of the disclosure, the biocompatible scaffold structure can comprise at least two polypeptides deposited thereon, and wherein one polypeptide can be fibrin deposited on the sheet of substantially parallel polymeric microfibers and at least one other polypeptide can be deposited on the fibrin.

In embodiments of this aspect of the disclosure, the biocompatible scaffold structure can further comprise a population of mesenchymal stem cells, or the progeny thereof.

In embodiments of this aspect of the disclosure, the biomimetic composition can comprise: a biocompatible scaffold structure comprising a sheet of substantially parallel polymeric microfibers, a population of hydroxyapatite nanoparticles distributed on the sheet in a pattern mimicking the mineralization of a native ligament to bone enthesis, fibrin deposited on said sheet of polymeric microfibers, at least one polypeptide deposited on the fibrin, wherein the at least one other polypeptide can be selected to promote the growth and/or differentiation of a population of mesenchymal stem cells or progeny thereof colonizing the scaffold structure, and a population of mesenchymal stem cells or progeny thereof, wherein the biocompatible scaffold structure is configured for replacing a native ligament of a subject animal or human.

In embodiments of this aspect of the disclosure, the biocompatible scaffold structure can be configured for replacing a native anterior cruciate ligament.

Another aspect of the disclosure encompasses embodiments of a method of forming a biomimetic scaffold structure, the method comprising the steps of: generating a sheet of substantially parallel polymeric microfibers having polymeric nanofibers deposited on the surface thereof; distributing hydroxyapatite nanoparticles on the sheet in a pattern mimicking the mineralization of a native ligament to bone enthesis; and configuring said sheet for replacing a native ligament of a subject animal or human.

In embodiments of this aspect of the disclosure, the method can further comprise contacting the sheet of substantially parallel polymeric microfibers with an alkali; providing exposed carboxyl groups; decreasing fiber diameter; and increasing surface roughness.

In embodiments of this aspect of the disclosure, the method can further comprise providing fibrin on the surface of the sheet of substantially parallel polymeric microfibers.

In embodiments of this aspect of the disclosure, the method can further comprise the step of colonizing the biomimetic scaffold with a population of mesenchymal stem cells or progeny.

In embodiments of this aspect of the disclosure, the method can further comprise the step of depositing a polypeptide on the fibrin on the surface of the sheet of substantially parallel polymeric microfibers, wherein the polypeptide is selected to promote the growth and/or differentiation of a population of mesenchymal stem cells or progeny thereof colonizing the biomimetic scaffold.

In embodiments of this aspect of the disclosure, the method of distributing hydroxyapatite nanoparticles on the sheet in a pattern mimicking the mineralization of a native ligament can comprise microprinting the hydroxyapatite nanoparticles onto the sheet of substantially parallel polymeric microfibers or electrophoretically depositing the hydroxyapatite nanoparticles, thereby forming a density gradient of the hydroxyapatite nanoparticles mimicking the mineralization of a native ligament to bone enthesis.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include ±1%, ±2%, ±3%, ±4%, ±5%, ±6%, ±7%, ±8%, ±9%, or ±10%, or more of the numerical value(s) being modified.

### EXAMPLES

### Example 1

### Production of an Aligned Electrospun PLA Nanofiber Mat

A 20% w/v solution of PLA with an intrinsic viscosity of 1.6 dL/g (Medisorb 100 L Poly(L-Lactide), Lakeshore Biomaterials, Birmingham, Ala.) using 1,1,1,3,3,3-hexafluoro-2-propanol (HFIP) as a solvent was prepared. Unidirectional electrospinning equipment was used; the PLA/HFIP solution was transferred to a 10 ml syringe affixed to a 20G×20 cm septum-penetrating needle connected to a voltage source, and held constant at 15 kV. The flow rate was established by a syringe pump set at 1 ml/h for 2 h.

Fibers were collected at a 15 cm distance on a 25 cm×15 cm sheet of aluminum foil attached via cellophane tape to a cylindrical aluminum mandrel 67 cm in diameter and 15.5 cm in length, rotating at 7000 rpm (24.6 m/s) with a stationary grounding point 3.5 cm behind the mandrel, as illustrated in FIGS. 2 and 5. Placing the grounding point behind the mandrel as opposed to grounding the mandrel yielded fiber uptake over a narrower and more consistent area (approximately 9 cm width perpendicular to the rotation axis compared to 15 cm when using a grounded mandrel).

After the electrospinning was complete, the resulting PLA fiber mat was not handled and left in the electrospinning chamber for 24 h to allow excess solvent to evaporate, thus avoiding curling or warping. The foil with the PLA fiber mat was then removed from the mandrel and placed in a vacuum desiccator environment for 72 h to evaporate residual HFIP and water. Due to the random deposition of fibers during the electrospinning process, the thickness of the fiber mat was inconsistent over the approximate 9 cm wide fiber uptake area. Specifically, the deposition would be greater towards the center of the samples, with decreasing thickness towards the outer edges. Multiple thickness measurements were taken across the transverse direction of the fibers from each sample (without the foil) using a thermomechanical analyzer (TMA); only the areas with a thickness range of between about 900 μm to about 1100 μm were used throughout the experimentation (FIG. 11). This typically resulted in two useable sections per PLA mat, of approximately 20 cm×2 cm.

### Example 2

### NaOH Treatment of Electrospun PLA

Electrospun PLA samples were placed into a glass beaker containing an aqueous solution of 0.25M NaOH at room temperature (21° C.). The samples were immersed for various time periods ranging from 1-40 min, most typically about 20 min, depending on the specific aim. Five samples were processed at a time and monitored to ensure complete immersion at all times and prevent clinging to each other and to the beaker wall. After their respective treatment times, the samples were removed from the NaOH solution and immediately immersed in a 500 ml beaker of deionized water to dilute any residual NaOH solution remaining on the samples. The samples were then rinsed 3 times with deionized water, placed on a low-lint paper cloth and dried in a desiccator for 72 h.

### Example 3

### Immobilization of Fibrin on the PLA

PLA samples were covered with a thin layer of fibrin to assess various parameters of each specific aim. Modifying methods to produce a homogenous surface layer of fibrin on a variety substrates (Campbell et al., (2005) Biomaterials 26: 6762-6770; Campbell et al., (1999) J. Biol. Chem. 274 30215-30221), incorporated herein by reference in their entireties, 50 ml of an aqueous solution containing 1.1 U/ml of bovine thrombin (Sigma Aldrich Co., St. Louis, Mo.) and 5 mm of calcium chloride was prepared and warmed to 37° C. Similarly, a 50ml aqueous solution containing 4 mg/ml of bovine fibrinogen (Sigma Aldrich Co., St. Louis, Mo.) and 50 mm of HEPES-buffered saline was prepared and warmed to 37° C. When combined, the thrombin/fibrinogen solution was fully polymerized into fibrin within 15 min. To effectively immobilize fibrin on the surface of the PLA samples, the thrombin and fibrinogen solutions were combined, immediately followed by immersion of the PLA. After a few seconds, the PLA samples were removed and placed in an incubator at 37° C. for 15 min, allowing the thrombin and fibrinogen to self-assemble into fibrin on the exposed surface of the PLA mat. The slides were then triple rinsed with PBS to remove unbound excess fibrin, and placed in a desiccator overnight to dry.

### Example 4

### Preparation of Samples for Cell Studies

The PLA fiber mat was cut into 15 mm×75 mm samples with the longer side parallel to the alignment of the fibers, and the foil was removed. The samples were then subjected to the various processing parameters necessary for each specific aim. The samples were then placed on individual 25 mm×75 mm glass slides, held in place by their static charge.

### Example 5

### Sample Fixation to the Tissue Culture Plates

Due to their thin size (approximately 1000 μm), flexibility, and high static attraction to almost every surface, handling the PLA samples without damaging them was difficult. Additionally, once immersed in an aqueous cell culture environment, the samples would float to the top of the surface, fold onto themselves along the fiber alignment direction, or cling to the sides of the cell culture well. However, for analysis of cell proliferation and alkaline phosphatase (ALP) activity, PLA samples were required that completely covered the bottoms of the culture plate wells; any human mesenchymal stem cell (hMSC) attachment to the bottom of the tissue culture-treated wells and subsequent proliferation/differentiation would impede the accuracy of the quantitative results.

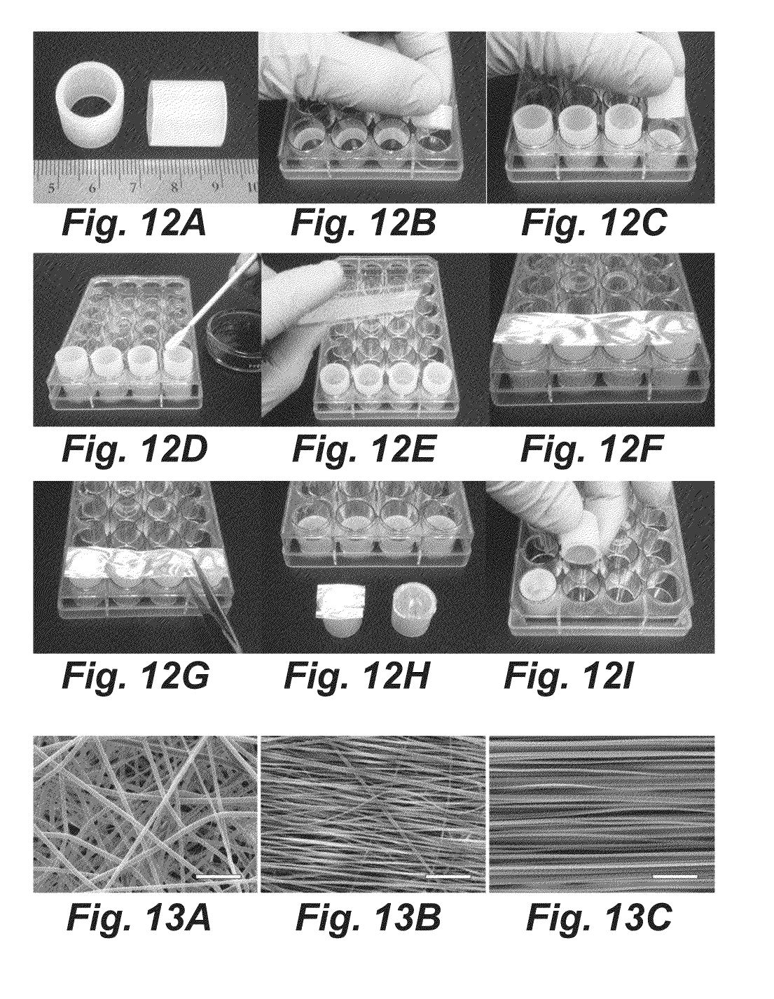

To facilitate a consistent means of obtaining a flat, continuous, and undamaged sample surface at the bottom of the culture well, the following sample fixation technique was implemented: Polyethylene (PE) tubing (LLDPE Value-Tube, Advanced Technology Products, Milford Center, Ohio) with 15.875 mm O.D and 12.7 mm I.D. was cut into 18 mm sections, as shown in FIG. 12A. The dimensions resulted in the PE sections fitting snugly in the wells of a 24-well cell culture plate (Beckton Dickinson and Co, Franklin Lakes, N.J.). One end of the tubes was ground flat and smooth using progressively finer grit sandpaper (240, 320, 400, and 600, respectively). The sections were washed with water, sonicated in a bath of acetone to remove any surface oils and ink product identification markings, followed by a 1 h soak in ethanol for sterilization.

To create a frame to hold the samples during preparation, a 24-well plate was modified by inserting 10 mm sections of the PE tubing at the base of four of the wells on one side of the plate to act as risers (see FIG. 12B). Four PE sections were inserted into the modified 24-well plate at a time (polished side up) and a biocompatible adhesive (Mastisol, Ferndale Laboratories, Inc., Ferndale, Mich.) was brushed onto the polished surface (FIGS. 12C and 12D). 15 mm×75 mm sections of the various PLA treatments were placed on a 25 mm×75 mm glass microscope slide (held by static charge), aligned with the four PE tubes, and pressed firmly and evenly on the tubes. This resulted in the effective transfer of the PLA section to the row of PE tubes (FIGS. 12E and 12F). The PLA was then cut with scissors between the PE tubes (FIG. 12G), and the separated tubes were removed from the well plate frame, individually “stamped” against a rubber surface to ensure complete adhesion of the PLA to the PE tube, and placed in a vacuum-desiccator to allow the adhesive to fully dry overnight. After the adhesive was dry, the PLA was trimmed around the PE tubes, the tubes were placed in sterile 24-well culture plates (FIGS. 12H and 12I). The modification resulted in flat PLA mats affixed to the bottom of the each of the wells with 1.26 cm² of useable surface area. All of the prepared 24-well plates were placed under a UV-light hood for 2 h, followed by rinsing the wells with sterilized deionized water 12 hours prior to seeding.

### Example 6

### Preparation of Samples for Imaging

To characterize the morphology of the hMSCs in response to the various PLA treatment/processing conditions, separate samples were prepared for SEM imaging following cell culture. Unlike the samples prepared for analysis of proliferation and ALP assessment, it was not essential that the PLA cover the entirety of the well bottom since only the surface of the PLA was observed for qualitative characterization of cell morphology. Samples of each of the PLA treatment conditions were cut into 1 cm² sections, and affixed to a 15 mm diameter round glass coverslip (Ted Pella Inc., Redding, Calif.) and allowed to dry in a vacuum desiccator overnight. The samples were then inserted face-up into sterile 12-well tissue culture plates and placed under a UV hood for 2 hours for sterilization, followed by rinsing the wells with sterilized deionized water 12 hours prior to seeding.

### Example 7

### Human Mesenchymal Stem Cell Culture

hMSCs were passaged, retaining only cells from passage numbers 4-6. The cells were cultured in mesenchymal stem cell basal medium (MSCBM) (Lonza, Inc. Walkersville, Md.) supplemented with MSCBM SINGLEQUOTS® (Lonza, Inc. Walkersville, Md.). Upon reaching confluence, cells were removed from the culture surface and deactivated by adding an equal volume of Dulbecco's modified eagle medium (DMEM) supplemented with 10% fetal bovine serum (FBS), 1% amphotericin B, 1% penicillin, 1% streptomycin, and 1% L-glutamine. The hMSCs were then centrifuged at 1,000 rpm for 5 min and re-suspended at a concentration of 15,000 cells per 677 μL of DMEM. The solution was measured into 677 μL aliquots and transferred to the 24-well cell culture plates with the PE tubes prepared for the study. The 12-well cell culture plates were also prepared for sample imaging.

Cell cultures were held constant under standard culture conditions (37° C., 95% relative humidity, 5% CO₂). Every 3-4 days, old media was replaced with 677 μL of fresh media. For the 24-well plate samples, the cells were cultured, trypsinized and harvested at day 1, 7, and 14, then cryogenically stored in Eppendorf tubes at −80° C. until analysis of cellularity was performed and alkaline phosphatase (ALP) activity assessed. For the 12-well plate imaging samples, the samples were fixed using 2.5% glutaraldehyde, and 2% paraformaldehyde in a sodium cacodylate buffer (0.2M, pH 7.4) with deionized water.

Following initial fixation, the samples were rinsed several times with PBS for a minimum of 15 min, followed by post-fixation with 1% sodium tetroxide in 0.1M phosphate buffer for 1 h. After re-rinsing with PBS several times for 15 min, the samples were dehydrated using a series of graded ethanol: 70% for 15 min, 95% for 15 min, and 3 changes of 100% for 10 min each. The samples were then subjected to chemical drying using 2 parts 100% ethanol and 1 part hexamethyldisilazane (HDMS) for 15 min, 1 part 100% ethanol and 2 parts HDMS for 15 min, then 2 changes of 100% HDMS for 15 min each. All residual solution was aspirated from the samples, and then the samples were allowed to air-dry under a fume hood overnight. The samples were then affixed to a mount, sputter-coated, and placed in the SEM. Images were acquired at an accelerating voltage of 5 kV.

### Example 8

### Analysis of Proliferation

Proliferation was analyzed using hMSCs harvested from the 24-well plates at d 1, 7, and 14. Picogreen assay (Molecular Probes, Eugene, Oreg.) was used to identify the double stranded DNA content according to manufacturer specifications. 100 μL cell extracts were placed in 96-well plates for analysis. Picogreen dye was added into the sample preparations then incubated in the dark for 15 min. Using a fluorescent microplate reader (Synergy HT, BIO-TEK Instruments, Winooski, Vt.) filtered at 485/528 (excitation/emission) was used to measure the double-strand DNA content.

### Example 9

Alkaline Phosphatase Activity: Quantitative ALP activity was assessed on the hMSCS harvested from the 24-well plates at 14 d. Using a fluorimetric SensoLyte FDP Alkaline Phosphatase Assay Kit (Anaspec, San Jose, Calif.), 50 μL aliquots of each sample were assayed for ALP content on a fluorescent microplate reader (using the same parameters as described in Example 8, above) then compared to a standard correlating known ALP content to fluorescence levels. A picogreen analysis was performed to normalize ALP expression via DNA content by determining the specific ALP activity present within each sample.

### Example 10

### Statistical Analysis

The results presented herein are representative data sets with experiments performed using six samples (n=6) for each condition at 1, 7, and 14 day time points. Values were expressed as ±standard error of mean (S.E.M.). Using SPSS software (SPSS, Chicago, Ill.), one-way analysis of variance (ANOVA) was performed to quantify any significant differences between conditions at each time point. Tukey multiple comparison tests were conducted to further determine significant differences between pairs. A value of p<0.05 was considered significant for all tests.

### Example 11

### Production of Electrospun PLA Mats with Altered Mandrel Velocity

For the purpose of evaluating mandrel rotation velocity on fiber diameter, alignment, and crystallinity, two PLA fiber mats were produced using the same parameters with the exception of altered mandrel rotation speed of 0 rpm (stationary) and 3500 rpm (12.3 m/s), respectively. These two PLA mats did not undergo any of the subsequent NaOH treatments.

### Example 12

Characterization of Fiber Alignment, Diameter, and Surface Morphology: SEM was used to analyze fiber alignment, diameter, and morphology of the PLA samples. Additionally, qualitative assessment of the surface morphology and binding efficacy of fibrin on PLA in the untreated vs. NaOH treated condition was evaluated using SEM. Sections of the 7000 rpm, 3500 rpm, stationary (0 rpm), and various NaOH-treated and fibrin-coated samples were affixed to a mount, sputter-coated with gold palladium and observed under SEM (Quanta 650FEG, FEI Co., Hillsboro, Oreg.) at an accelerating voltage of 5 kV. The SEM micrographs were analyzed to measure fiber diameter utilizing image-analyzing software.

### Example 13

### Assessment of Crystallinity by Differential Scanning Calorimetry

DSC analyses were performed on PLA samples of the following morphology: as received (pellet), thin film (cast from the electrospinning solution), unaligned electrospun fibers (0 rpm), aligned electrospun fibers (7000 rpm), and 20 min NaOH treated aligned electrospun fibers (7000 rpm). The degree of crystallinity for each sample was calculated using the following formula:

% Crystallinity=(ΔHexp/ΔH100)×100

where ΔHexp and ΔH100 are the melting enthalpy values for the experimental sample and a fully crystalline sample, respectively.

### Example 14

### Swelling Study

To evaluate physical changes that may have occurred under physiological conditions over time, the PLA in the untreated and NaOH treated conditions were placed in a PBS solution to evaluate swelling (PBS uptake) over various time periods. A PLA fiber mat was cut into multiple 1 cm² samples and the foil was removed. Half of the samples were subjected to the 20 min NaOH treatment protocol. Samples of both conditions (untreated/NaOH treated) were placed into separate 25 ml glass vials containing PBS solution (pH 7.4), sealed, and incubated at 37° C. for 1, 7, 14, 30, and 60 d. At the end of each interval, the samples were removed from the PBS, dabbed on a lint-free paper towel to remove surface PBS, and immediately weighed to obtain wet mass. The samples were then placed in a desiccator for 72 h to dry thoroughly. The samples were removed from the desiccator and immediately weighed to identify dry mass. Percent swelling was calculated using the following equation:

% Swelling=(Mw−Md)/Md×100

where, Mw is the wet mass after the various incubation periods, and Md is the mass after the sample has been dried. All samples were run in triplicate (n=3) for statistical validity. This method to measure swelling was modified from K. S (2008) J. Oral Tissue Engineering 6: 77-87, incorporated herein by reference in its entirety for measuring PLA degradation over time.

### Example 15

### Sectioning and Measurement of the Electrospun PLA Mats

To obtain consistent samples for accurate mechanical testing, the PLA fiber mats were cut into 5 mm×45 mm sections, with the 45 mm length parallel to the alignment of the fibers. Following any surface treatment performed, the thickness of each sample was measured in the different locations along the sample width by the TMA, and averaged to calculate cross-sectional area for tensile testing. Mechanical Testing of the PLA Nanofiber Mats:

A micromechanical testing system (Minimat Model 2000, TA Instruments, New Castle, Del.) was used on the various samples to establish mechanical behavior under uniaxial tension. The distance between the clamps was set at 25 mm. Sections of adhesive-backed 320 grit sandpaper were affixed to the contact surfaces of the test clamps to provide better contact and minimize slipping of the bulk fibers under load. The samples were placed in the clamps, and centered with calipers (±0.25 mm) to ensure uniaxial loading along the fiber alignment direction prior to tightening of the clamps. A heated chamber was used for some of the samples to establish mechanical properties at temperatures up to 37° C. (normal human body temperature). Testing was performed in tension mode with a load cell of 200N and at a strain rate of 5 mm/min until failure. The mechanical properties of the samples were then defined based on these uniaxial data obtained.

### Example 16

### Optimization of a Hydroxyapatite Nanoparticle (Nanohydroxyapatite. nanoHA) Bioink

An aqueous solution containing 5% w/v of hydroxyapatite nanoparticles (Nanocerox Inc., Ann Arbor, Mich.), 11% v/v propylene glycol (C₃H₈O₂) (used as a dispersant and humectant), and 85% v/v deionized water was mixed, and then sonicated to 2 h to break up hydroxyapatite nanoparticles agglomerates. The average size of the hydroxyapatite nanoparticles in the solution was characterized using dynamic light scattering. An additional batch of the hydroxyapatite nanoparticles bioink (not used for any ensuing cell studies) was produced with the addition of 1% w/v of a fluorescent dye (calcein) for the purpose of characterizing printed patterns under fluorescent microscopy.

The hydroxyapatite nanoparticles bioinks were loaded into a cartridge, and placed into bioinkjet printer (JetLabII, Microfab Technologies Inc, Plano, Tex.) with a 50 μm diameter nozzle. This type of piezoelectric printer permits drop-on demand control of the hydroxyapatite nanoparticles bioink deposition, with multiple jetting parameters that can be customized to fine tune droplet volume, velocity, and firing frequency. Pattern parameters such as droplet spacing and overlap could be controlled, and multiple arrays with variable dimensions could be written in script form and uploaded to the device for printing. Two CCD cameras provided feedback on the jetting parameters: droplets firing out of the nozzle and drop deposition on the target substrate, respectively.

### Example 17

### Design of a Printed Gradient Pattern

The ligament-to-subchondral bone transition in the ACL spans a width of 1400 μm based on mechanical heterogeneity. Proximal to the ligament end of that interface, the fibrocartilage covers an approximate 300 μm distance with roughly 150 μm of mineralization, so the total distance of mineralized tissue (mineralized fibrocartilage and subchondral bone) across the interface is approximately 1250 μm. Using that dimension as a reference, multiple pattern-programming scripts were written to determine the most effective means of replicating a gradient of mineralization as seen in the ACL.

By adjusting such parameters as droplet spacing and droplet overlay, a variety of gradients with a height of 1250 μm were printed on a silicon wafer and imaged with the CCD camera on the bioprinter. The printed patterns were compared to histological images of the ACL-bone interface (enthesis), resulting in the selection of the gradient with the most similar anatomical profile.

### Example 18

### Inkjet Printing of Hydroxy Apatite Nanoparticles on the PLA Nanofibers

Electrospun PLA fiber mats were cut into 15 mm×75 mm samples with the longer side parallel to the alignment of the fibers. To optimize the mechanical and morphological properties, the samples were subjected to a 20 min NaOH treatment, and then placed in a desiccator for 72 h. The samples were then placed on individual 25 mm×75 mm glass slides, held in place by their static charge. The glass slides with the 15 mm×75 mm NaOH treated PLA samples were loaded onto the stage of the bioinkjet printer. Four identical 1.25 mm×10 mm gradient patterns were printed onto the aligned PLA fibers, with the gradient variation parallel to the fibers to replicate the anatomical orientation of an ACL microstructure.

### Example 19

### Characterization of the Printed Hydroxyapatite Nanoparticles

SEM coupled with EDS was used to characterize the distribution of hydroxyapatite nanoparticles on the PLA fibers via printing. A section of the PLA mat with the printed hydroxyapatite nanoparticles gradient was affixed to a mount, sputter-coated with gold-palladium and placed in the SEM. The sample was examined and imaged using an accelerating voltage of between 5-15 kV, depending on magnification (due to beam damage on the PLA fibers). Spectra of mineral distribution were obtained using energy dispersive X-ray analysis (EDS) (INCA Energy 200 EDS, Oxford Instruments, Oxfordshire, UK) at 200× magnification and an accelerating voltage of 20 kV to determine Ca and P content. The spectra were collected over 200 μm square regions over various areas of the 1250 μm printed hydroxyapatite nanoparticles gradient pattern, as well as areas beyond the gradient without any visible hydroxyapatite nanoparticles droplets to serve as a control.

### Example 20

### Stability Evaluation of the Printed Hydroxyapatite Nanoparticles Gradient

Due to various aqueous processing and experimental conditions (immobilization of fibrin, cell culture, etc.), the hydroxyapatite nanoparticles immobilized on the surface of the NaOH-treated PLA fibers were evaluated to assess their stability in an aqueous environment. PLA samples were printed with the hydroxyapatite nanoparticles-calcein bioink to produce the gradient. The samples were cut in half along the direction of the aligned fibers at the center of the gradient. One of the halves of the samples were immersed in separate 25 mL vials containing a solution of phosphate buffered saline (pH 7.4), and incubated at 37° C. for 72 h, changing the PBS daily. Following the 72 h incubation, the samples were taken out and placed in a desiccator for 72 h. The hydroxyapatite nanoparticles-calcein samples were affixed to a microscope slide and imaged under fluorescence microscopy to qualitatively compare any changes in the gradient morphology.

### Example 21

### Immobilization and Characterization of Fibrin on the PLA

A portion of the hydroxyapatite nanoparticles printed samples were coated with fibrin to evaluate any encapsulation effects on the printed particles and gauge hMSCs response as a result of the combination the fibrin and hydroxyapatite nanoparticles. SEM micrographs were obtained to qualitatively assess any modification of surface morphology and encapsulation of the hydroxyapatite nanoparticles in fibrin on the surface of the PLA fibers.

### Example 22

### Fiber Alignment, Diameter, and Morphology

PLA nanofibers were produced utilizing electrospinning. Fibers were collected on three uptake conditions: stationary (0 rpm), 3500 rpm mandrel rate, and 7000 rpm mandrel rate. SEM images exhibited nanofibrous morphology for all conditions with varying degrees of inter-fiber spacing, diameter, and alignment (FIGS. 13A-13C). The stationary sample had relatively straight fibers that were randomly oriented with an average diameter of 2608±353 nm (FIG. 13A). The 3500 rpm sample showed a more ordered alignment and orientation perpendicular to the axis of mandrel rotation, reduced inter-fiber spacing compared to the stationary sample, and an average fiber diameter of 1396±312 nm (FIG. 13B). As the uptake rate increased to 7000 rpm, the fibers achieved a highly-aligned morphology perpendicular to the axis of mandrel rotation. The inter-fiber spacing was significantly reduced in comparison to the stationary and 3500 rpm samples. Similarly, the average fiber diameter was reduced to 760±96 nm with greater regularity (FIG. 13C). These findings indicate that increased uptake rates for electrospun PLA resulted in increased fiber order and alignment, and the rates were inversely proportion to fiber diameter and spacing.

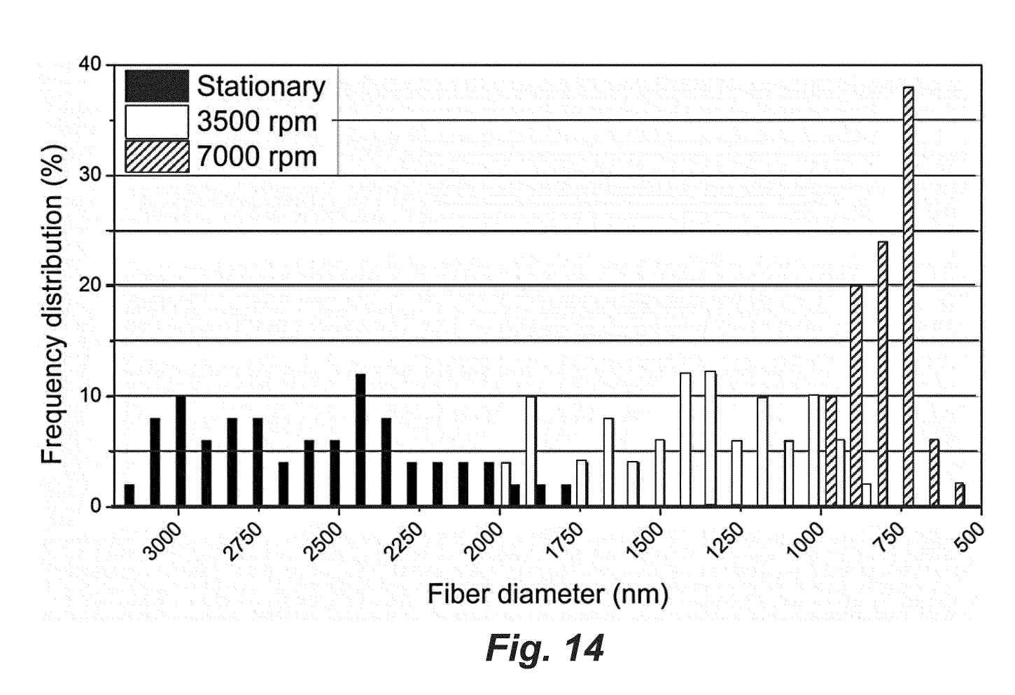

Average fiber diameters ranged from about 760 nm to about 2608 nm depending on the uptake condition. The range of fiber diameters within a specific uptake condition was also uptake-rate dependent. FIG. 14 shows the frequency distribution of fiber diameters for the electrospun PLA at the different uptake conditions. Increased uptake rates resulted in decreasing distributions of the fiber diameters. This was shown with the stationary, 3500 rpm, and 7000 rpm rates and their standard error of mean (S.E.M.) (n=50) distributions of ±50, ±44, and ±14, respectively.

To facilitate a fiber diameter mimetic of the 50-500 nm diameter collagen bundles, the PLA electrospun at the 7000 rpm rate was subjected to 0.25M NaOH hydrolysis treatments of 1, 5, 10, 20, and 40 min time intervals. Table 1 shows the average diameter and reduction in cross-sectional fiber area as a result of the NaOH treatments.

TABLE 1
NaOH treatment effects on 7000 rpm PLA nanofibers.

| | Average | |
|---|---|---|
| NaOH Treatment | diameter (nm) ± | Reduction in fiber area (%) ± |
| Time (min) | S.E.M* | S.E.M* |
| 0 (control) | 760 ± 13.6 | — |
| 1 | 700 ± 13.6 | 13.6 ± 3.4 |
| 5 | 601 ± 11.1 | 36.4 ± 2.3 |
| 10 | 564 ± 12.3 | 43.5 ± 2.3 |
| 20 | 457 ± 12.5 | 62.5 ± 2.1 |
| 40 | 399 ± 13.1 | 70.9 ± 2.0 |

*n = 50

The 1, 5 and 10 min treatments all had fiber diameters above the upper limit of the collagen bundles. Treatments at 20 and 40 min produced fibers that fall within the upper range of the collagen bundles with average diameters of 457±89 nm and 399±92 nm, respectively. However, the 40 min fibers were found to be very brittle and would separate easily during handling, while the 20 min samples maintained a structural integrity more similar to the untreated samples.

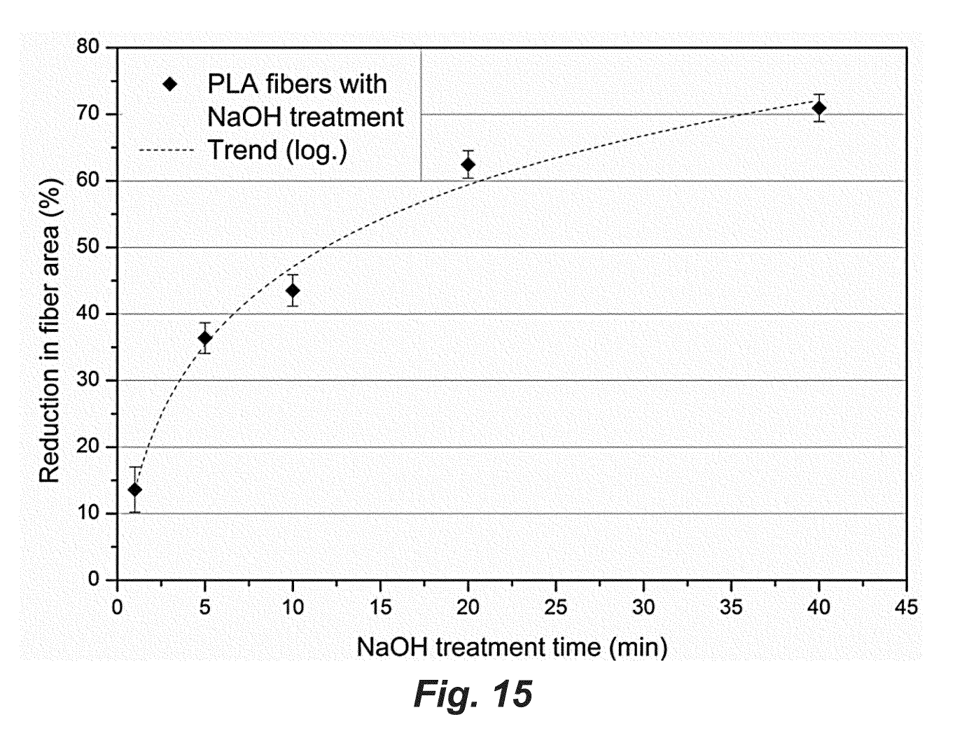

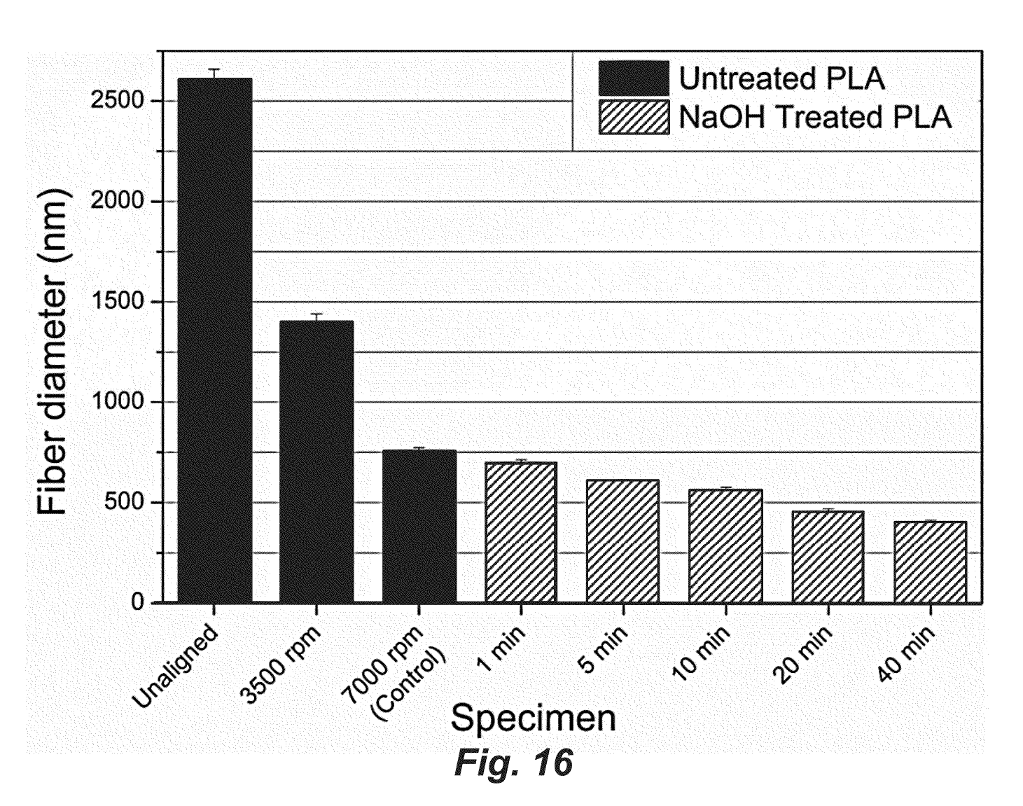

The final cross-sectional area of the fibers was shown to be dependent of NaOH treatment time, with the percent reduction in area following a logarithmic trend as treatment time was increased, as shown in FIG. 15. The combined effects of increasing the electrospun PLA uptake rate followed by increasing intervals of hydrolytic degradation treatments on fiber diameter are illustrated in FIG. 16.

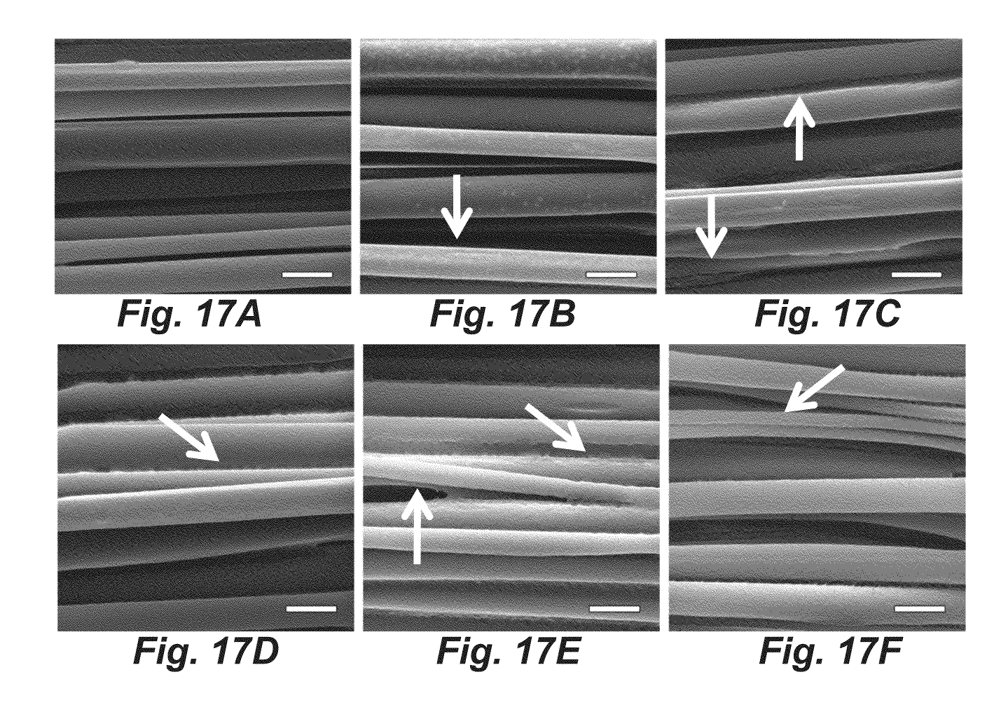

The various NaOH treatments also had an effect on the surface morphology of the fibers, as shown in FIGS. 17A-17F. The control sample (FIG. 17A) had a smooth and continuous surface morphology. There were some small cracks (less than 100 nm) present at certain points on the fibers, believed to be caused by expansion/contraction of the polymer and subsequent cracking of the gold-palladium coating during sample preparation, or by thermal damage from the electron beam during SEM imaging. The fibers displayed no pitting or altered shape/thickness along the length of the fibers. The 1 min sample displayed relatively smooth fiber morphology with a very minor amount of pitting and surface roughness, indicated by the arrow in FIG. 17B.

There were lightly colored spots on the fibers that were found throughout all of the 1 min samples, believed to be points of increased surface roughness. When observed at the visible edge of a fiber, there appeared to be a dip in surface topography. Attempts at viewing the spots at a higher magnification were not possible due to thermal damage caused by the electron beam on the polymer when magnification was increased.

The 5 and 10 min samples in FIGS. 17C and 17D, respectively, exhibited more pronounced degradation compared to the control and 1 min samples. A higher degree of surface roughness could be observed, and there was visible pitting at the interface between fibers, indicated by the arrows. The 20 and 40 min samples displayed a degree of surface roughness and interface pitting, as indicated by the arrows in FIGS. 17E and 17F, respectively. Additionally, a reduced overall fiber diameter was visibly apparent when compared to the control sample. These data presented indicate that obtaining PLA nanofibers via unidirectional electrospinning with a mandrel rotational velocity of about 24.6 m/s, followed by a 20 min treatment in 0.25M NaOH solution will result in a polymer matrix with fiber alignment and diameter mimetic of native collagen bundles found in human ligament tissue.

### Example 22

Analysis of Crystallinity: Using an intrinsic viscosity value of 1.6 dL/g, the molecular weight of the PLLA used in this study was calculated to be 56,273 using the Mark-Houwink equation in the following form:

[η]=5.45×10⁻⁴·Mᵥ^{0.73}

where η and Mᵥ^{0.73} are the intrinsic viscosity and average molecular weight, respectively. To calculate crystallinity, the value of 75.57 J/g was used, corresponding to the DSC thermogram of a fully crystalline sample of PLLA with a similar molecular weight as the PLLA used herein. The results of the DSC analysis are shown in Table 2.

TABLE 2
Differential scanning calorimetry (DSC) data of various PLA morphologies

| | | | | | Electrospun |
|---|---|---|---|---|---|
| | As | | Electrospun | Electrospun | Fibers |
| | Received | Solution Cast | Fibers | Fibers | (Aligned, 20 min |
| Sample | (Pellet) | Thin Film | (Random) | (Aligned) | NaOH treated) |
| Melting | 60.58 | 34.06 | 41.54 | 54.14 | 54.62 |
| Enthalpy | | | | | |
| (J/g) | | | | | |
| Melting | 178.72 | 174.93 | 174.74 | 174.17 | 174.55 |
| Temperature | | | | | |
| (° C.) | | | | | |
| Crystallization | 80.16 | 45.07 | 54.97 | 71.64 | 72.28 |
| (%) | | | | | |

The various morphologies had little effect on the melting temperatures. However, the melting enthalpy was significantly reduced with the solution cast compared to the as received sample. Melting enthalpy and subsequent crystallization increased when the PLA solution was electrospun into randomly oriented fibers. Crystallinity was further increased when the fibers were collected on the 7000 rpm rotating mandrel. The reduced diameters of the aligned fibers indicate a significant degree of fiber stretching, and subsequently, strain-induced crystallization. The 20 min NaOH treated aligned fibers displayed a slight increase in crystallinity compared to the untreated aligned fibers, likely due to polymer chain scission caused by the NaOH hydrolysis and the subsequent reduction in molecular weight on the fiber surface.

### Example 23

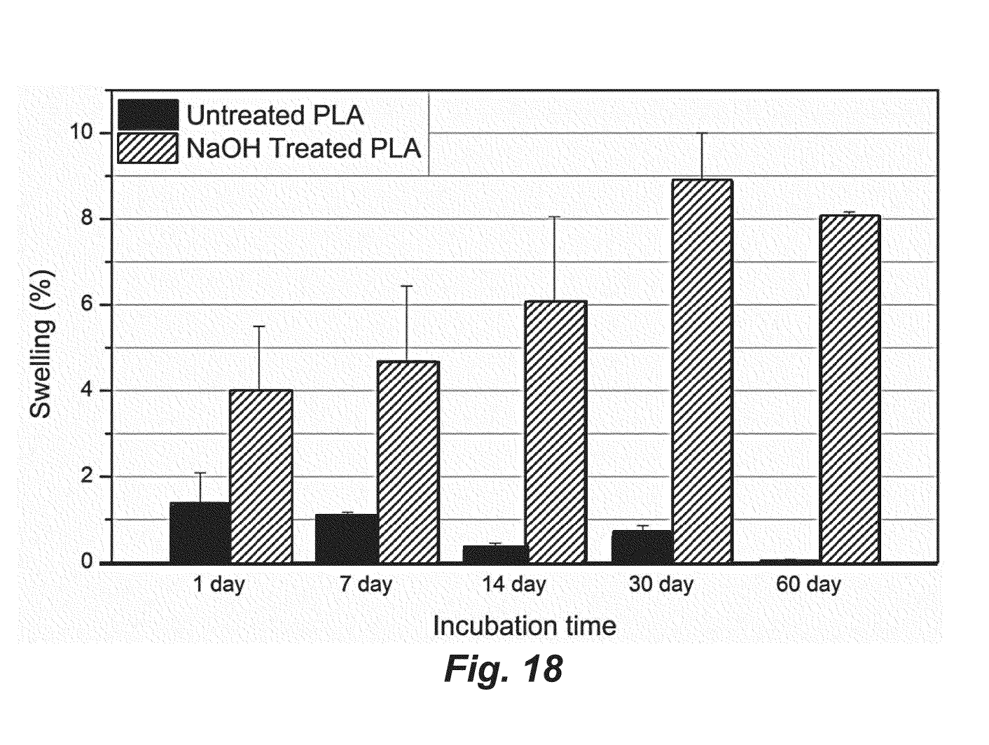

Swelling: FIG. 18 displays the percent swelling of the PLA fibers in the untreated and NaOH treated conditions over a 60-day period. Samples in the untreated condition displayed minimal swelling (0.07% to 1.1%), likely due to the high hydrophobicity of PLA. Conversely, the NaOH treated samples displayed modest swelling (4.04% to 9.2%) due to their increased surface roughness and surface carboxyl groups. It is presumed that this effectively reduces hydrophobicity and increases PBS uptake.

### Example 24

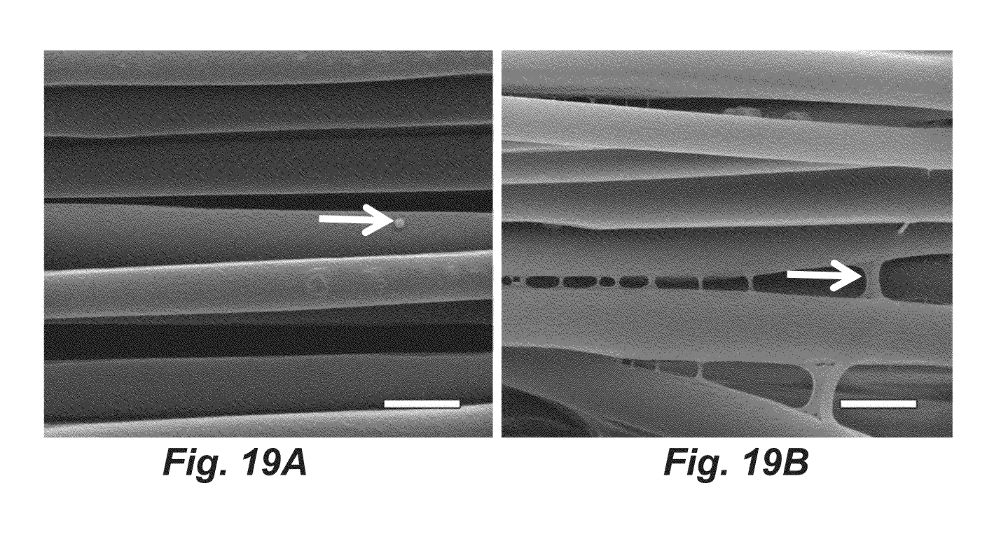

Immobilization of Fibrin: Samples of the 7000 rpm PLA in the untreated and 20 min NaOH treatment condition were subjected to surface immobilization of fibrin. As shown in FIGS. 19A and 19B, there was a difference in the surface morphology of the untreated PLA (FIG. 19A) and the NaOH treated sample (FIG. 19B). The surfaces of the untreated fibers were smooth, continuous, and closely resembled the control sample shown in FIG. 17A, with the exception of particles randomly scattered along the surface, presumably comprised of fibrin (indicated by an arrow in FIG. 19A).

Conversely, the NaOH treated sample displays a different morphology from the untreated sample with the added fibrin, as well as the sample with the same NaOH treatment in FIG. 17E. The pitting and rough surface topography seen in the samples with the same 20 min NaOH treatment was eliminated and was generally smooth with an apparently confluent superficial layer of fibrin. Another indicator of confluence was the connecting membranes at the interface between fibers, indicated by the arrows in FIG. 19B. It is considered that the highly hydrophobic nature of the untreated PLA may inhibit effective adhesion of fibrin, but when hydrolytically degraded by NaOH, the subsequent addition of the carboxyl groups and increase in surface roughness results in an increased binding affinity of fibrin.

### Example 25

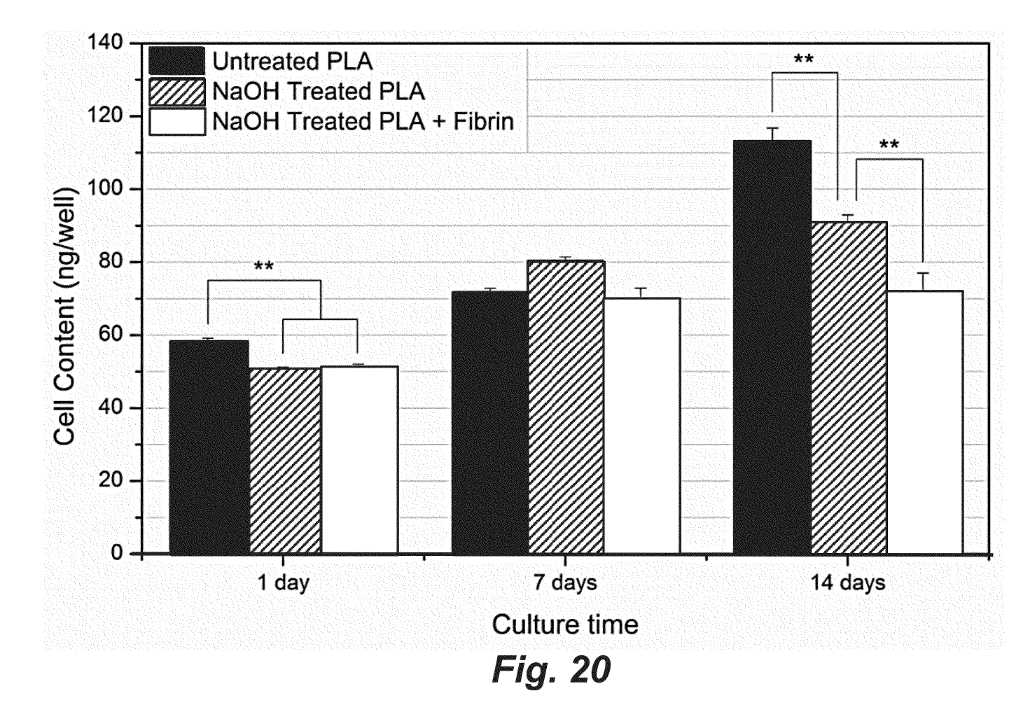

Cell Studies: To determine their proliferative rate over time, hMSCs were cultured on three conditions of the PLA fiber mats (untreated, 20 min NaOH, and 20 min NaOH+fibrin) and their long term proliferation was assessed over 14 d, as shown in FIG. 20. By day 14, cellular proliferation had progressed in a fashion highlighting a decrease in proliferative rate based on modifications to the PLA fiber mats. Proliferation proceeded most rapidly on the untreated PLA fiber mats, approaching a cellular DNA content of 113.13±3.66 ng/well. Cells cultured on 20 min NaOH-treated PLA mats displayed slightly less proliferation over time. The addition of fibrin onto the 20 min NaOH-treated PLA mat further slowed proliferation, showing little increase in cellularity between d 1 and 14. Considering that proliferation is known to plateau at the onset of differentiation, it is possible that NaOH treatment modified the PLA surface properties in a manner that facilitated osteogenic differentiation and that the addition of fibrin does so further.

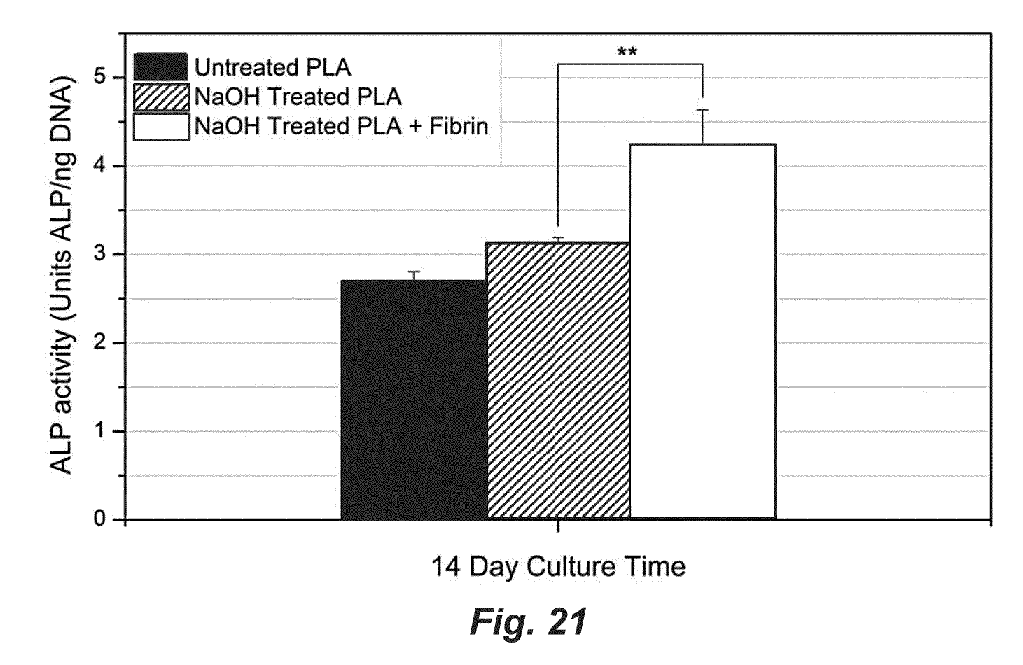

To assess mineralization, hMSCs were cultured on the modified PLA fiber mats for up to 14 d, following which physical ALP activity was determined utilizing a quantitative biochemical assay (FIG. 21). These values were normalized by DNA content as a means to assess the activity of each individual cell as opposed to total ALP activity. By day 14, the 20 min NaOH+fibrin condition exhibited statistically greater ALP activity relative to the other conditions.

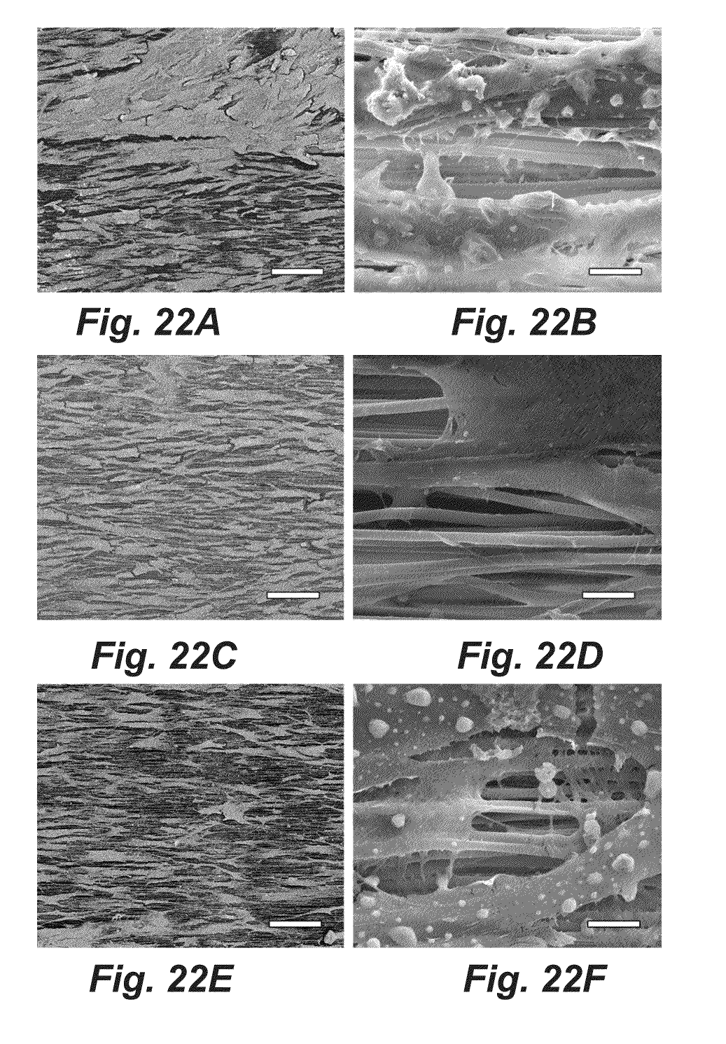

SEM images of the hMSCs on the various PLA samples at day 14 are displayed in FIGS. 22A-22E. FIG. 22A displays the untreated PLA sample at 250× magnification; an almost confluent layer of cells can be seen on the sample with individual cells displaying an elongated and spindle-like morphology, aligned in the general direction of the PLA fibers. A higher magnification (50,000×) image of the same sample is shown in FIG. 22B. The PLA fibers are visible with the cells exhibiting finger-like projections extending towards the fibers. FIG. 22C displays the 20 min NaOH treated sample with a visible cell density slightly less than seen on the untreated sample and a higher degree of cell alignment, but with similar cell morphology. The higher magnification image corresponding to the 20 min NaOH sample is shown in FIG. 22D.

There is not a significant amount of discernible difference at high magnification between the untreated sample and the 20 min NaOH sample. FIG. 22E shows the fibrin coated NaOH-treated sample. There is a reduction in cell density on the surface compared to the other samples at that magnification. The morphology of some of the cells appears to be slightly less elongated or spindle-like, adopting a more rectangular or cuboidal shape.

The high magnification image shows small interconnecting strings of fibrin between the PLA fibers and more pronounced cellular processes integrating with the fibers and fibrin. The various images in FIGS. 22A-22E correlate to the quantitative data obtained from the analysis of proliferation and the ALP study; the untreated sample had a visibly higher quantity of cells on the surface with a diminishing amount for the NaOH-treated and the NaOH+fibrin-treated samples, respectively. Additionally, a subtle change in morphology (from elongated/spindle-like to shorter/more cuboidal) is visible in the fibrin coated sample when compared to the others, indicating the early onset of osteogenic differentiation. The cells spread and oriented themselves along the direction of the fibers. This is advantageous in an engineered ligament. An aligned orientation of cells and collagen fibers occurs in native ligament tissue. Subsequent differentiation, and the ECM production by the hMSCs on the scaffolds of the disclosure can follow the same morphology due to the presence of the aligned PLA fibers.

Based on the results, osteogenic differentiation appears to have been enhanced by the 20 min NaOH treatment and even further by the addition of fibrin as indicated by decreased proliferation and enhanced ALP activity. This behavior being exhibited on the NaOH treatment conditions is likely based on an increase in surface roughness since surface topography has been shown to modify cellular behavior, as described earlier. The addition of fibrin led to even further enhanced osteogenic activity.

During NaOH hydrolysis, carboxyl groups were added to the exposed surface of the PLA fibers and surface roughness was visibly increased. The affinity of the fibrin and improved cellular response to the hydrolyzed fibers may have been a function of surface roughness, surface carboxyl groups, or both.

### Example 26

Uniaxial tensile tests of the aligned electrospun PLA fibers were performed using ten samples (5 dry, 5 wet) per surface treatment condition including a control (unmodified aligned electrospun sample). The data revealed considerable variety in the mechanical properties based on the surface treatments employed on the samples. The wet samples did not deviate beyond the range of the data obtained from the dry samples of each given condition. One-way ANOVA performed on the wet/dry samples for each treatment condition yielded p-values ranging from 0.12-0.28, indicating relatively equal sample means. Therefore, the data obtained for the wet and dry samples were combined. Numerical results are reported as mean±standard error of mean (S.E.M.).

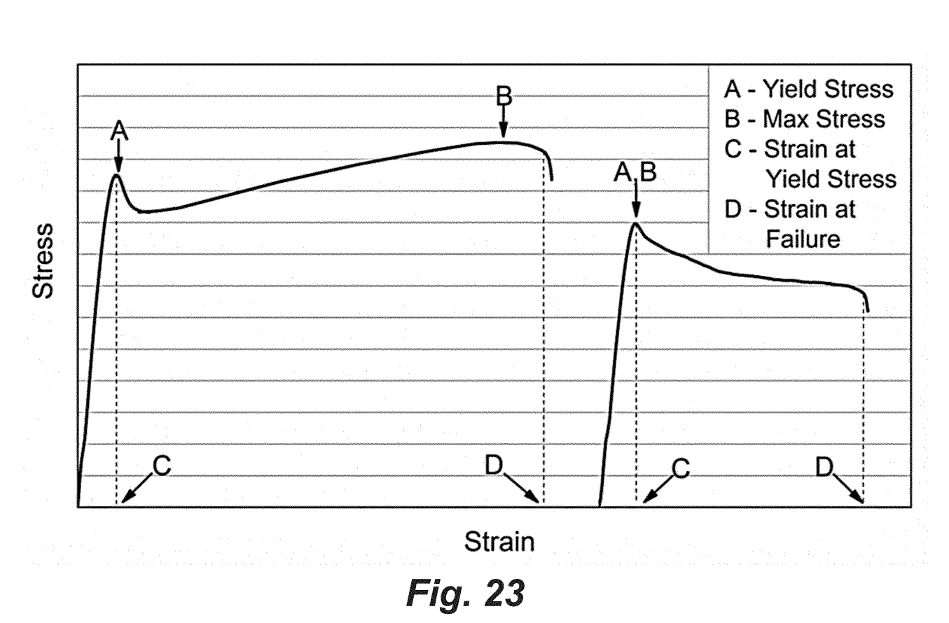

FIG. 23 displays a representation of the two conditions obtained throughout the mechanical study. The yield stress (A) is the point at which the linear elastic region yields and begins to exhibit non-linear deformation. The maximum stress is the highest value of stress obtained throughout the test, and can differ from the yield stress due to strain-induced crystallization of the polymer fibers (B). The strain at yield stress is the percent elongation at the end of the linear elastic region (C), and the strain at failure is the percent elongation when the fibers begin to rupture, quantitatively assessed in this study by the intersection of a downward 45° line tangent to the stress/strain curve at the onset of failure (D). This method was implemented because a consistent means of establishing the point of total failure was difficult, due to the stair-step shape of the stress-strain curve during failure as fibers were breaking irregularly rather than in unison.

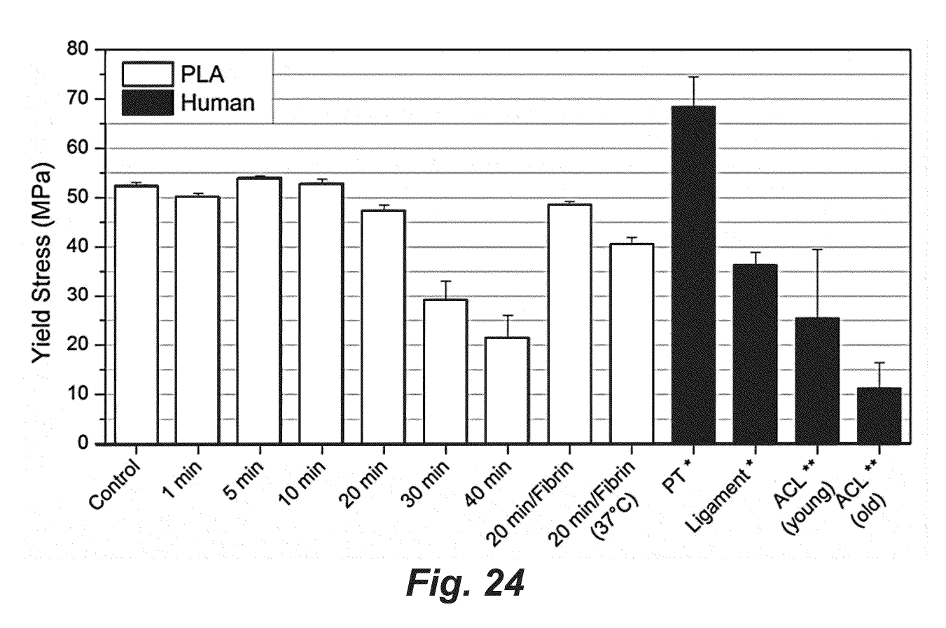

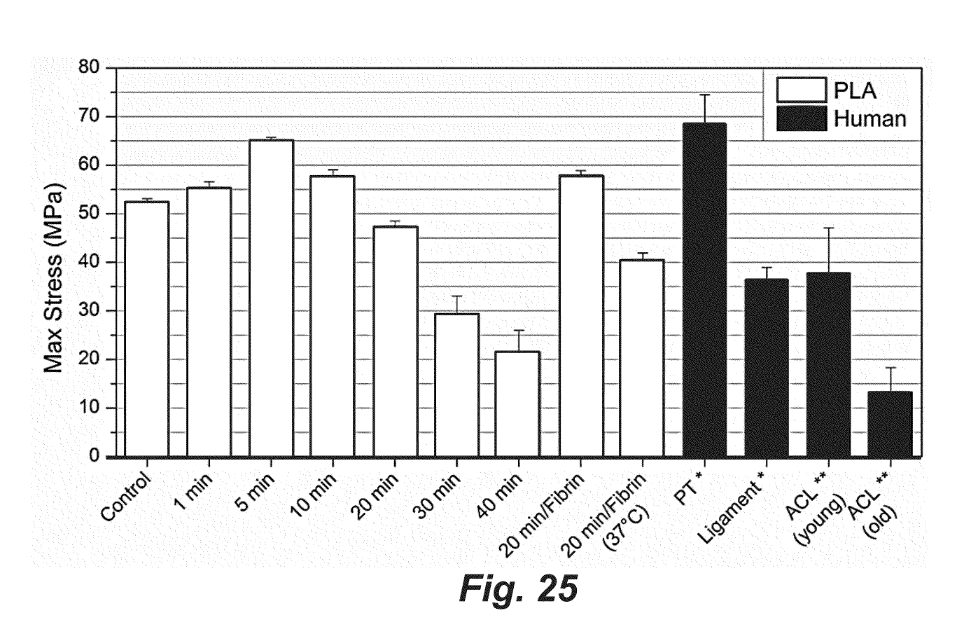

The yield and maximum stresses for the samples are illustrated in FIGS. 24 and 25, respectively. The 1 min samples indicated a slight loss of yield strength (50.1 MPa±0.78), while 5 and 10 min samples displayed slightly higher yield strength than the control sample, with values of 54.1 MPa±0.29, 53.0 MPa±0.79, and 52.5 MPa±0.59, respectively.

The variety of 20 min samples (20 min, 20 min+fibrin, and 20 min+fibrin at 37° C.) exhibited a modest decline in yield stress (47.4 MPa±1.1, 48.6 MPa±0.66, and 40.6 MPa±1.3, respectively) and displayed properties most similar to the ACL and ligament tissue data, particularly the 20 min+fibrin at 37° C. sample. The 30 min and 40 min samples had a sharp decline in yield stress with a wider distribution of values (29.2 MPa±3.9 and 21.5 MPa±4.5, respectively).

The maximum stress values were higher than the yield stress for the 1, 5, 10, and 20 min+fibrin samples (55.4 MPa±1.2, 65.2 MPa±0.48, 57.8 MPa±1.2, and 58.0 MPa±0.89, respectively). For the 1, 5, and 10 min samples, strain induced crystallization is likely responsible for the increase in strength caused by the hydrolytic degradation by NaOH and subsequent lowering of the molecular weight on the PLA fiber surface, corresponding to an increase in degree of crystallization during strain (Kulkarni et al., (2007) Surface and Interface Analysis 39: 740-746). In terms of simple crystallization kinetics, a shorter polymer chain (lower molecular weight) can align into ordered lamellae easier than a longer chain.

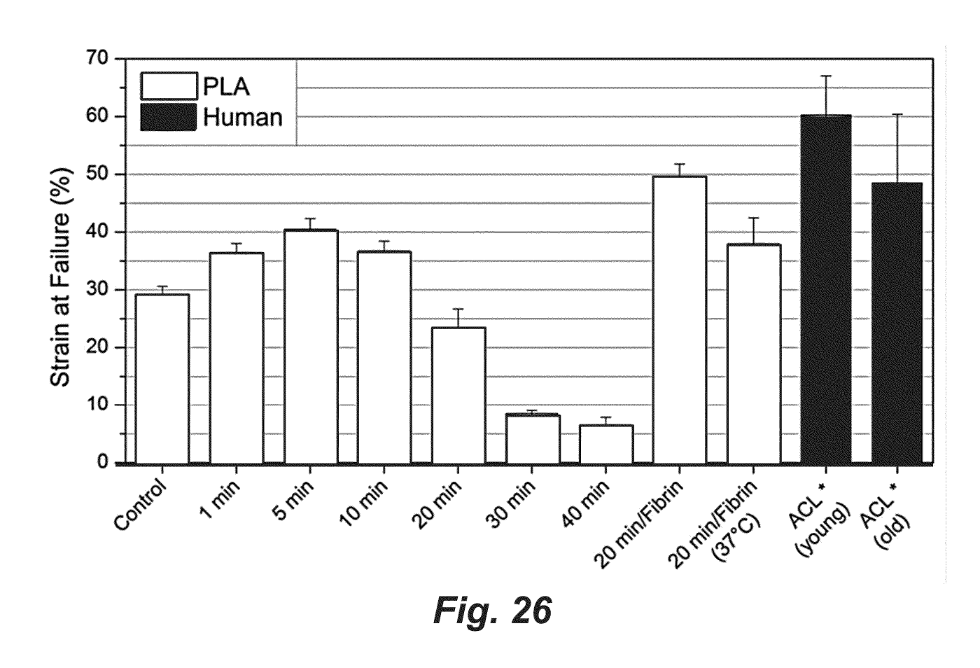

FIG. 26 displays the percent elongation at failure, with the 1 min, 5 min and 10 min NaOH-treated sample values higher than the control sample (36.4%±1.6, 40.5%±1.8, 36.7%±1.7, and 29.2%±1.4, respectively), which corresponds to the aforementioned strain induced crystallization observed for the same samples. The 20 min, 30 min and 40 min samples (23.3%±3.3, 8.4%±0.60, and 6.5%±1.4, respectively) exhibited elongation at failure less than the control sample, with the 30 min and 40 min samples failing shortly after the onset of plastic deformation with less than 10% elongation at failure. The highest values of strain at failure were observed for the 20 min+fibrin (49.6%±2.2) and 20 min+fibrin at 37° C. (38.0%±4.5) samples, both within range of natural ACL tissue.

A combination of factors can result in a preferred response, where increased strength caused by decreased molecular weight (due to hydrolysis) is not affected by reduced strength caused by reduced fiber diameter (also due to hydrolysis). The 5 min and 20 min+fibrin samples both display these properties and are both within the range of the natural ACL and graft tissue for yield/maximum stress. The 5 min sample had higher stresses (yield and maximum) and a greater percent elongation at failure than all of the other NaOH-treated samples. The 20 min+fibrin sample also displays a maximum stress higher than the yield stress, believed to be caused by a combination of strain-induced crystallization kinetics and the large extensibility of fibrin. However, the percent elongation at failure for the 20 min+fibrin samples is the highest out of all the samples tested, and it is within the range of natural ACL tissue.

The addition of fibrin on the surface of the fibers fortified the reduced diameter caused by the 20 min NaOH-treated PLA, effectively negating the effects of lowered yield and maximum stresses and the strain at failure, when compared to the 20 min sample without fibrin.

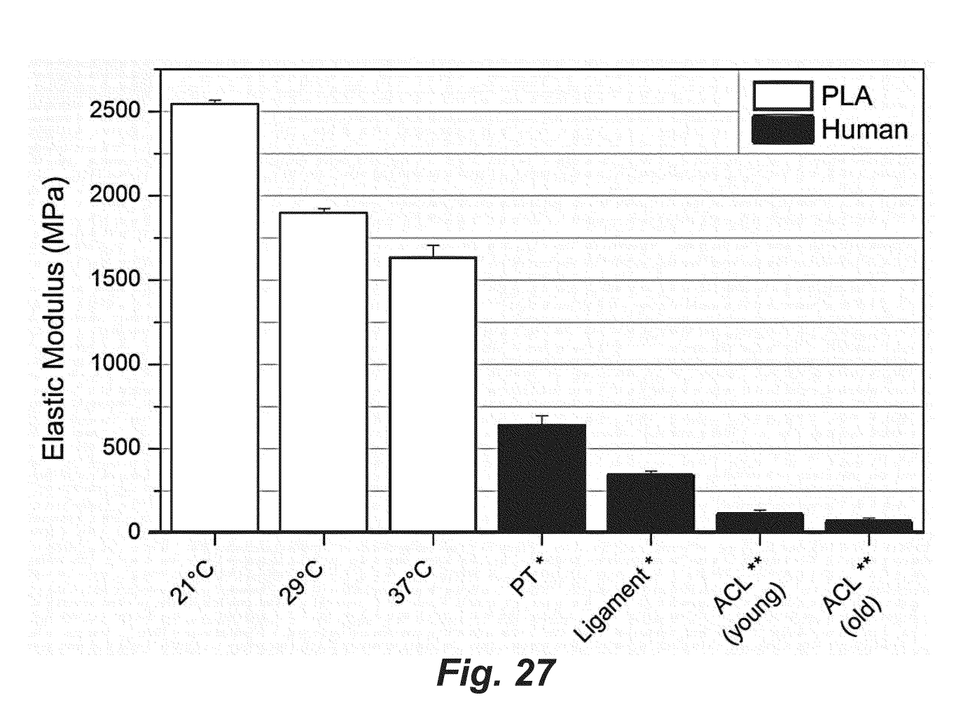

The elastic modulus for the PLA was consistent (2542 MPa±23) for all of the samples tested (n=80) at room temperature; however, the values were about 4-fold higher than the values of the patellar tendon (643 MPa±53) and between 7- to 40-fold higher than the various ligament data (65-345 MPa), as seen in FIG. 27. Samples were also tested at 29° C. and 37° C. to see the effects of temperature on the modulus, yielding 1884 MPa±39 and 1636 MPa±70, respectively. The 37° C. (body temperature) sample was approximately 2.5-fold higher than the patellar tendon; however, it indicated a 36% reduction in modulus compared to samples tested at room temperature.

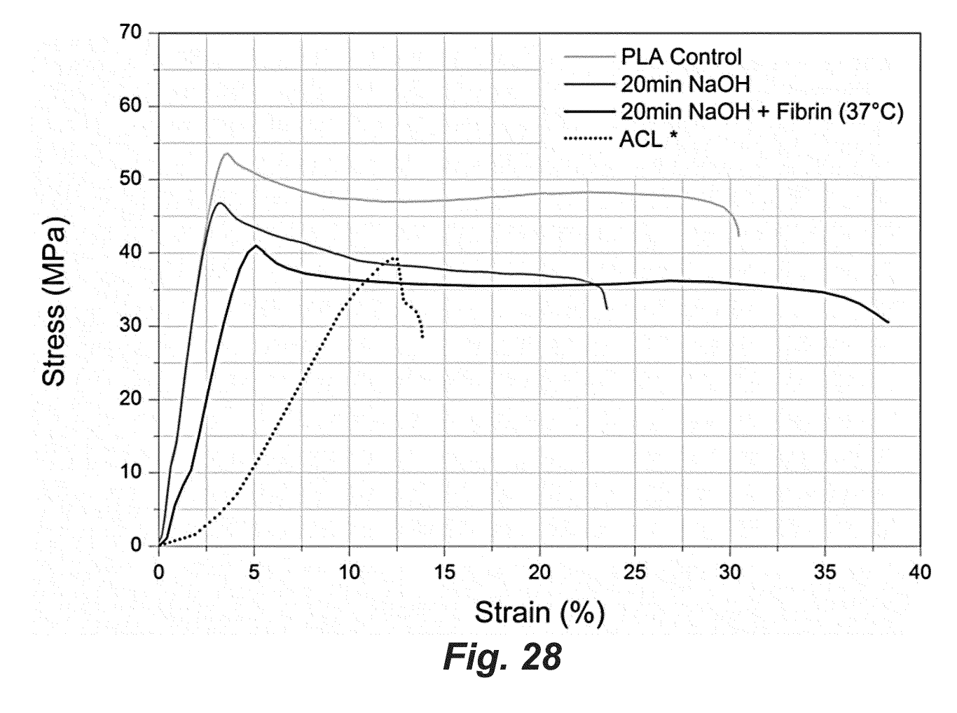

To visually understand the overall effects of the various surface treatments and temperature on the mechanical behavior of the PLA fibers, FIG. 28 displays the stress-strain profiles of a control, 20 min NaOH-treated sample, and 20 min NaOH-treated sample with fibrin at body temperature. The stepwise process of electrospinning a highly aligned PLA nanofiber mat, followed by a 20 min NaOH hydrolysis treatment, then immobilizing fibrin on the hydrolyzed surface of the fibers results in a mechanical profile more similar to human ligament tissue than unmodified PLA.

What is claimed:

1. A biomimetic composition comprising:
a biocompatible scaffold structure comprising a sheet of substantially parallel polymeric microfibers and a population of hydroxyapatite nanoparticles deposited on the sheet, wherein the population of hydroxyapatite nanoparticles is distributed on the sheet in a pattern mimicking the mineralization of a native ligament to bone enthesis.

2. The biomimetic composition of claim 1, wherein the biocompatible scaffold structure is biodegradable.

3. The biomimetic composition of claim 1, wherein the polymeric microfibers comprise poly(lactic acid).

4. The biomimetic composition of claim 1, wherein the biocompatible scaffold structure further comprises at least one polypeptide deposited thereon.

5. The biomimetic composition of claim 4, wherein the at least one polypeptide is selected from the group consisting of: an extracellular matrix polypeptide, fibrin, fibrinogen, a cell growth factor, and a cell differentiation inducer.

6. The biomimetic composition of claim 4, wherein the at least one polypeptide deposited thereon is fibrin.

7. The biomimetic composition of claim 5, wherein the biocompatible scaffold structure comprises at least two polypeptides deposited thereon, and wherein one polypeptide is fibrin deposited on the sheet of substantially parallel polymeric microfibers and at least one other polypeptide is deposited on the fibrin.

8. The biomimetic composition of claim 1, wherein the biocompatible scaffold structure further comprises a population of mesenchymal stem cells, or the progeny thereof.

9. The biomimetic composition of claim 1, comprising:
a biocompatible scaffold structure comprising a sheet of substantially parallel polymeric microfibers;
a population of hydroxyapatite nanoparticles distributed on the sheet in a pattern mimicking the mineralization of a native ligament to bone enthesis;
fibrin deposited on said sheet of polymeric microfibers;
at least one polypeptide is deposited on the fibrin, wherein the at least one other polypeptide is selected to promote the growth and/or differentiation of a population of mesenchymal stem cells or progeny thereof colonizing the scaffold structure; and
a population of mesenchymal stem cells or progeny thereof,
wherein the biocompatible scaffold structure is configured for replacing a native ligament of a subject animal or human.

10. The biomimetic composition of claim 9, wherein the biocompatible scaffold structure is configured for replacing a native anterior cruciate ligament.

11. A method of forming a biomimetic scaffold structure, the method comprising the steps of:
generating a sheet of substantially parallel polymeric microfibers having polymeric nanofibers deposited on the surface thereof;
distributing hydroxyapatite nanoparticles on the sheet in a pattern mimicking the mineralization of a native ligament to bone enthesis; and
configuring said sheet for replacing a native ligament of a subject animal or human.

12. The method of claim 11, further comprising contacting the sheet of substantially parallel polymeric microfibers with an alkali; providing exposed carboxyl groups; decreasing fiber diameter; and increasing surface roughness.

13. The method of claim 11, further comprising providing fibrin on the surface of the sheet of substantially parallel polymeric microfibers.

14. The method of claim 11, further comprising the step of colonizing the biomimetic scaffold with a population of mesenchymal stem cells or progeny.

15. The method of claim 13, further comprising the step of depositing a polypeptide on the fibrin on the surface of the sheet of substantially parallel polymeric microfibers, wherein the polypeptide is selected to promote the growth and/or differentiation of a population of mesenchymal stem cells or progeny thereof colonizing the biomimetic scaffold.

16. The method of claim 11, wherein the method of distributing hydroxyapatite nanoparticles on the sheet in a pattern mimicking the mineralization of a native ligament comprises microprinting the hydroxyapatite nanoparticles onto the sheet of substantially parallel polymeric microfibers or electrophoretically depositing the hydroxyapatite nanoparticles, thereby forming a density gradient of the hydroxyapatite nanoparticles mimicking the mineralization of a native ligament to bone enthesis.
